(19) United States
(12) Patent Application Publication
(10) Pub. No.: US 2016/0214983 A1
(43) Pub. Date: Jul. 28, 2016

(54) 3,7-DIAZABICYCLO[3.3.1]NONANE CARBOXAMIDES AS ANTITHROMBOTIC AGENTS

(71) Applicant: COUNCIL OF SCIENTIFIC & INDUSTRIAL RESEARCH, New Delhi (IN)

(72) Inventors: Dinesh Kumar DIKSHIT, Lucknow (IN); Anil Kumar KARUNAKARAN SASIKALA, Lucknow (IN); Madhu DIKSHIT, Lucknow (IN); Manoj Kumar BARTHWAL, Lucknow (IN); Ankita MISRA, Lucknow (IN); Manish JAIN, Lucknow (IN)

(21) Appl. No.: 15/025,864

(22) PCT Filed: Jul. 9, 2014
(86) PCT No.: PCT/IN2014/000458

§ 371 Date: Mar. 29, 2016

(30) Foreign Application Priority Data
Sep. 30, 2013 (IN) 2892/DEL/2013

(51) Int. Cl. C07D 471/08

(52) U.S. Cl. CPC C07D 471/08

(57) ABSTRACT

The present invention relates to the 3,7-diazabicyclo[3.3.1]nonane carboxamides and process for preparation thereof. The present invention further relates to the compounds of general formula 1 possessing anti-thrombotic (anti-platelet) activities. The invention also relates to use of these moieties as inhibitors of collagen induced platelet adhesion and aggregation mediated through collagen receptors both in vitro and in vivo. Further, invention also relates these class of compounds exhibiting anti-platelet efficacy through dual mechanism inhibited both collagen as well as U46619 (thromboxane receptor agonist) induced platelet aggregation.

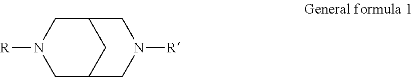

wherein, R′ is;

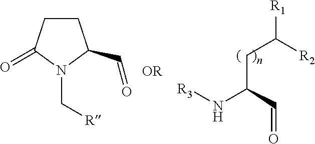

wherein R is selected from alkyl, acyl, tosyl, tert-butyloxycarbonyl, araalkyl or substituted araalkyl groups; R″ is selected preferably from halogen, cyano, lower alkyl, aryl, substituted aryl, and tosyl groups; R1 is selected from hydrogen and lower alkyl groups; R2 is selected from lower alkyl and aryl groups; R3 is selected from tert-butyloxycarbonyl and bezyloxycarbonyl groups; n=0,1.

### FIELD OF THE INVENTION

The present invention relates to the substituted 3,7-diazabicyclo[3.3.1]nonane (commonly known as bispidine) carboxamides based molecules as antithrombotic (anti-platelet agents) agents. The present invention also relates to the use of these moieties as inhibitors of collagen induced platelet adhesion and aggregation mediated through collagen receptors. Further, the present invention also relates this class of compound exhibiting anti-platelet efficacy through dual mechanism inhibited both collagen as well as U46619 (thromboxane receptor agonist) induced platelet aggregation. The present invention further relates to the process and preparation of substituted 3,7-diazabicyclo[3.3.1]nonane (commonly known as bispidine) carboxamides based molecules.

### BACKGROUND OF THE INVENTION

The curiosity in the designing of cyclic diamine scaffold stems from the finding of nipecotamide analogs as platelet aggregation inhibitors induced by ADP (Lasslo A et. al., Med. Prog. Technol. 1986; 11: 109; Folie B J et. al., Blood, 1989; 72: 1393), collagen (Lasslo A et. al., Am. Soc. Art. Int. Organs 1983; 6: 47), thrombin (Petrusewicz J et. al., Biochim. Biophys. Acta 1989; 983: 161), epinephrine (Gollamudi R et. al., Thromb. Haemostas. 1993; 69: 1322) and the stable TxA2 mimetic in vitro (Gollamudi R et. al., Thromb. Res. 1993; 69: 361).

Amides of N-substituted pyroglutamic acids have been reported as moderate inhibitor of thrombin (Dikshit et al, 2001 Indian Patent 1206/DEL/2001) and have shown anti-thrombotic activity in mice model of thrombosis. Watson et al used the amides of piperidine and the more lipophilic bispidine unit to prepare N-substituted pyrrolidine analogues (Fig. A) as potent, selective factor Xa inhibitor with good anticoagulant activity (Nigel S Watson et. al., Bioorganic & Medicinal Chemistry Letters 2006; 16: 3784-3788). Further, N-acetylated bispidine derived compounds (Fig. B) were found to be useful in the treatment of cardiac arrhythmias (U.S. Pat. No. 6,887,881 BI). Moreover, many amino acid and peptidyl derivatives having 3 or 4-aminomethyl-1-amidinopiperidine were reported as potential antithrombotics (U.S. Pat. No. 6,255,301).

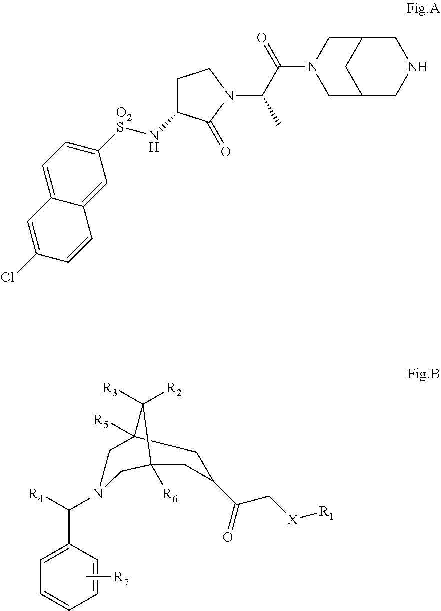

With the recognition that a high frequency of treatment failures occur with single anti-platelet therapy, there has been a strong push for the routine use of more intensive anti-platelet therapy that includes Aspirin and Clopidogrel. However, individuals receiving the therapy reportedly suffer from bleeding risk, thereby prompting a reevaluation of antithrombotic regimens that can maximize efficacy without increasing the risk of bleeding. A great deal of insight has been gained into the contribution of collagen, thromboxane A₂ (TxA₂) and their respective receptors and signaling mechanism in promoting platelet adhesion, activation and subsequent thrombus growth and stability. Hence, targeting against the synergy between collagen and TxA₂ mediated platelet activation pathway could prove to be novel and very useful in terms of improving the outcome of high intensity antithrombotic therapy.

Considering the structural features of nipecotamides and the highly promising activity of pyroglutamic acid derived amides synthesized in our laboratory, the proposed work focuses to introduce rigidity in the nipecotamide by incorporating them in bicyclic diamine framework, and to put various acyl, alkyl and aryl residues at the 3^{rd} and 7^{th} nitrogens of bispidine. Further, we also proposed bispidine acylated with some protected, hydrophobic amino acids, expecting enhanced activity. Confirmationally rigid systems such as bispidines can provide required orientation to the molecules so that it could easily arrange itself to interact with the enzyme and prevent the hydrophobic collapse.

### OBJECT OF THE INVENTION

The main object of the present invention is to provide 3,7-diazabicyclo[3.3.1]nonane carboxamides of general formula 1 and process for preparation thereof.

Another object of the present invention is to provide compounds of formula 1, having significant anti-thrombotic activity both in vivo and in vitro.

Further object of the invention is to relate this class of compound of formula 1, exhibiting anti-platelet efficacy through dual mechanism inhibiting both collagen as well as U46619 (thromboxane receptor agonist) induced platelet aggregation.

### SUMMARY OF THE INVENTION

Accordingly, the present invention provides a compound of general formula 1;

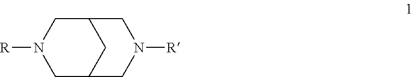

wherein, R′ is;

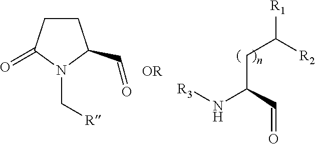

wherein R is selected from alkyl, acyl, tosyl, tert-butyloxycarbonyl, araalkyl or substituted araalkyl groups; R″ is selected preferably from halogen, cyano, lower alkyl, aryl, substituted aryl and tosyl groups; R₁ is selected from hydrogen and lower alkyl groups; R₂ is selected from lower alkyl and aryl groups; R₃ is selected from tert-butyloxycarbonyl and bezyloxycarbonyl groups; n=0,1

In an embodiment of the present invention, the representative compounds of general formula 1 comprising;
1. tert-butyl 7-(1-Benzyl-5-oxo-pyrrolidine-2-carbonyl)-3,7-diaza-bicyclo[3.3.1]nonane-3-carboxylate, (1a)
2. tert-butyl 7-[1-(2-Bromo-benzyl)-5-oxo-pyrrolidine-2-carbonyl]-3,7-diaza-bicyclo[3.3.1]nonane-3-carboxylate, (1b)
3. 1-Benzyl-5-(7-benzyl-3,7-diaza-bicyclo[3.3.1]nonane-3-carbonyl)-pyrrolidin-2-one, (1c)
4. (5S)-5-(7-Benzyl-3,7-diaza-bicyclo[3.3.1]nonane-3-carbonyl)-1-(2-bromo-benzyl)-pyrrolidin-2-one, (1d)
5. tert-butyl 7-[1-(4-Methyl-benzyl)-5-oxo-pyrrolidine-2-carbonyl]-3,7-diaza-bicyclo[3.3.1]nonane-3-carboxylate, (1e)
6. (5S)-5-(7-Benzyl-3,7-diaza-bicyclo[3.3.1]nonane-3-carbonyl)-1-(4-methyl-benzyl)-pyrrolidin-2-one, (1j)
7. (5 S)-5-(7-benzyl-3,7-diazabicyclo[3.3.1]nonane-3-carbonyl)-1-(2,6-dichlorobenzyl) pyrrolidin-2-one, (1g)
8. (5S)-5-(7-benzyl-3,7-diazabicyclo[3.3.1]nonane-3-carbonyl)-1-(4-chlorobenzyl)pyrrolidin-2-one, (1h)
9. (5 S)-5-(7-benzyl-3,7-diazabicyclo[3.3.1]nonane-3-carbon yl)-1-tosylpyrrolidin-2-one, (1i)
10. tert-butyl 7-((S)-1-(4-cyanobenzyl)-5-oxopyrrolidine-2-carbonyl)-3,7-diazabicyclo[3.3.1]nonane-3-carboxylate, (1j)
11. tert-butyl 7-((S)-1-(4-chlorobenzyl)-5-oxopyrrolidine-2-carbonyl)-3,7-diazabicyclo[3.3.1]nonane-3-carboxylate, (1k)
12. tert-butyl 7-((S)-1-(2,6-dichlorobenzyl)-5-oxopyrrolidine-2-carbonyl)-3,7-diazabicyclo[3.3.1]nonane-3-carboxylate, (1l)
13. tert-butyl 7-((S)-1-(4-methoxybenzyl)-5-oxopyrrolidine-2-carbonyl)-3,7-diazabicyclo[3.3.1]nonane-3-carboxylate, (1m)
14. tert-butyl 7-((S)-1-(naphthalen-1-ylmethyl)-5-oxopyrrolidine-2-carbonyl)-3,7-diazabicyclo [3.3.1]nonane-3-carboxylate, (1n)
15. (5S)-5-(7-benzyl-3,7-diazabicyclo[3.3.1]nonane-3-carbonyl)-1-(4-bromobenzyl)pyrrolidin-2-one; (1o)
16. (5S)-5-(7-benzyl-3,7-diazabicyclo[3.3.1]nonane-3-carbonyl)-1-(4-methoxybenzyl)pyrrolidin-2-one, (1p)
17. (5 S)-5-(7-Benzoyl-3,7-diaza-bicyclo[3.3.1]nonane-3-carbonyl)-1-(2-bromo-benzyl)-pyrrolidin-2-one, (1q)
18. 1-(2-Bromo-benzyl)-5-[7-(toluene-4-sulphonyl)-3,7-diaza-bicyclo[3.3.1]nonane-3-carbonyl]-pyrrolidin-2-one, (1r)
19. (5S)-5-(7-benzyl-3,7-diazabicyclo[3.3.1]nonane-3-carbonyl)-1-tosylpyrrolidin-2-one, (1s)
20. (5S)-5-(7-Benzoyl-3,7-diaza-bicyclo[3.3.1]nonane-3-carbonyl)-1-(4-methyl-benzyl)-pyrrolidin-2-one, (1t)
21 (5 S)-5-(7-(2-bromobenzyl)-3,7-diazabicyclo[3.3.1]nonane-3-carbonyl)-1-(4-methylbenzyl) pyrrolidin-2-one, (1u)
22. (5S)-5-(7-(4-bromobenzyl)-3,7-diazabicyclo[3.3.1]nonane-3-carbonyl)-1-(4-methylbenzyl) pyrrolidin-2-one, (1v)
23. (5S)-5-(7-(4-chlorobenzyl)-3,7-diazabicyclo[3.3.1]nonane-3-carbonyl)-1-(4-methylbenzyl) pyrrolidin-2-one, (1w)
24. benzyl (2S)-1-(7-benzyl-3,7-diazabicyclo[3.3.1]nonan-3-yl)-3-methyl-1-oxobutan-2-yl carbamate, (1x)
25. benzyl (2S)-1-(7-benzyl-3,7-diazabicyclo[3.3.1]nonan-3-yl)-4-methyl-1-oxopentan-2-yl carbamate, (1y)
26. benzyl (2S)-1-(7-benzyl-3,7-diazabicyclo[3.3.1]nonan-3-yl)-1-oxo-3-phenylpropan-2-yl carbamate, (1z)

In still another embodiment of the present invention, the compounds of generals formula 1 are useful as anti-thrombotic agents (antiplatelets agents) via collagen-epinephrine induced pulmonary thromboembolism in mice (in vivo) and collagen induced platelet aggregation in human platelets (in vitro).

In yet another embodiment of the present invention, the % protection of compounds of general formula 1, by collagen plus epinephrine induced pulmonary thromboembolism in mice (in vivo) varies from 25 to 60% at 30 μM concentration.

In still another embodiment of the present invention a process for preparation compound of general formula 1

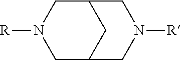

wherein, R′ is;

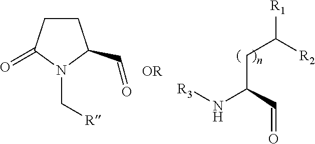

wherein R is selected from alkyl, acyl, tosyl, tert-butyloxycarbonyl, araalkyl or substituted araalkyl groups; R″ is selected preferably from halogen, cyano, lower alkyl, aryl, substituted aryl, and tosyl groups; R₁ is selected from hydrogen and lower alkyl groups; R₂ is selected from lower alkyl and aryl groups; R₃ is selected from tert-butyloxycarbonyl and bezyloxycarbonyl groups; n=0,1, comprising the steps of:
i) reacting a first compound with a second compound to obtain a reaction mass comprising compound of general formula 1 and more particularly, one or more of compound of formula 1a to 1p and 1x to 1z, wherein the first compound being selected from
   (a) a compound of general formula

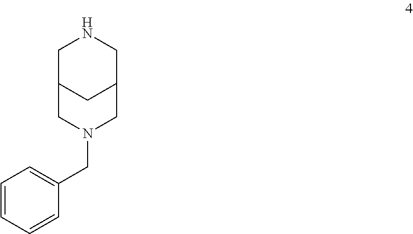

or
(b) a compound of general formula

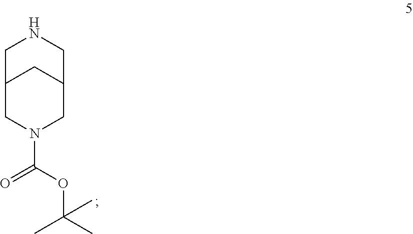

and
the second compound being selected from a group comprising of (a) a compound of general
formula

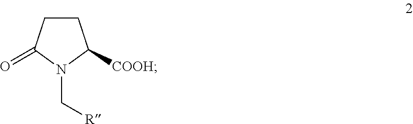

or (b) a compounds of general formula

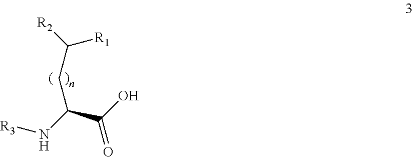

wherein, R″ is selected preferably from halogen, cyano, lower alkyl, aryl, substituted aryl, and tosyl groups; R₁ is selected from hydrogen and lower alkyl groups; R₂ is selected from lower alkyl and aryl groups; R₃ is selected from tert-butyloxycarbonyl and bezyloxycarbonyl groups; n=0,1; with the proviso that the compound of general formula 2 is reaction with compounds of general formula 4 and 5, both; and the compound of general formula 3 is reacting with compound of general formula 4 only,
ii) if the reaction mass thus obtained in step (i) comprises one or more compound of formula 1a, 1b, 1e, 1j to 1n, then, deprotecting a Boc-Group in the reaction mass with TFA at a temperature ranging between 0° C. to 15° C. for a period in the range of 4 to 5 hours followed by N-acylation at temperature ranging between 0° C. to 25° C. in solvent selected from DCM or THF followed by N-benzylation at temperature ranging between 50 to 60° C. for a period in the range of 4 to 5 hours in acetone to obtain a reaction mass comprising deprotected compound of formula 1q to 1w and converting the deprotected compound of formula 1q to 1w thus obtained to N-benzylation, benzoylation, tosylation to provide protected compound 1q to 1w;
wherein:
a) the compounds of formula 1a to 1p include:

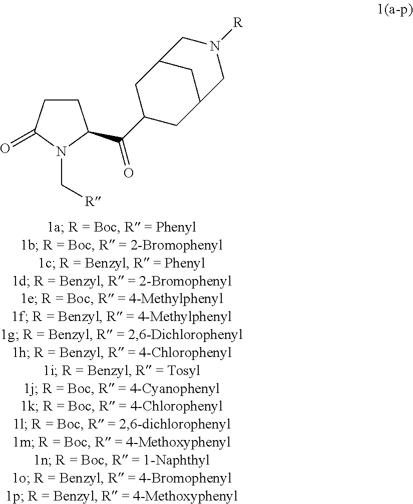

with R″ is selected from halogen, cyano, lower alkyl, aryl, substituted aryl, tosyl and naphthyl groups; R is selected from alkyl, aryl, tert-butyloxycarbonyl or substituted araalkyl groups;
b) the compounds of formula 1x to 1z include:

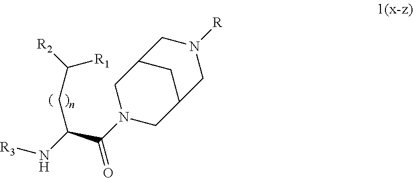

with R is selected from alkyl, tert-butyloxycarbonyl, araalkyl or substituted araalkyl groups; R₁ is selected from hydrogen and lower alkyl groups; R₂ is selected from lower alkyl and aryl groups; R₃ is selected from tert-butyloxycarbonyl and bezyloxycarbonyl groups; n=0,1; and
c) the compounds of formula 1q to 1w include:

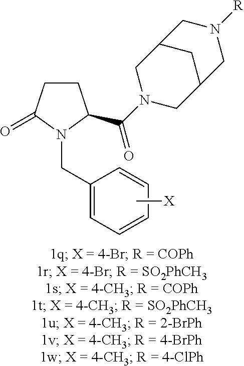

with R is selected from acyl, tosyl, or substituted araalkyl groups; X is selected preferably from halogen, and lower alkyl groups.

In yet another embodiment of the present invention, the reaction of step (i) takes place in the presence of a coupling agent selected from the group consisting of dicyclohexylcarbodiimide, benzotriazole-1-yl-oxy-tris-pyrrolidino-phosphonium hexafluorophophate, isobutyl chloroformate-TEA/DIPEA, oxalyl chloride-TEA/DIPEA or an activating agent 1-hydroxy benzotrizole at a temperature ranging between −20° C. to 0° C. for a period in the range of 30 to 45 min, followed by stirring at temperature range from 25-30° C. for a period ranging from 2-3 hours in aprotic solvents selected from DCM, THF and dioxane.

In yet another embodiment of the present invention, N-benzylation in step (ii) of the process for the preparation of general formula 1, is carried in dry acetone in presence of anhydrous potassium carbonate (K₂CO₃) followed by the addition of substituted benzyl bromide by refluxing at a temperature ranging 50-60° C. for 2-3 hours.

In still another embodiment of the present invention, benzoylation in step (ii) of the process for the preparation of general formula 1, is carried in dry dichloromethane using benzoyl chloride in presence of triethylamine or diisopropylethyl amine at a temperature ranging from 0-5° C. for 30-60 minutes.

In yet another embodiment of the present invention, tosylation in step (ii) of the process for the preparation of general formula 1, is carried in dry dichloromethane using toluenesulphonyl chloride in presence of triethylamine or diisopropylethyl amine at a temperature ranging from 0-5° C. for 30-60 minutes.

In still another embodiment of the present invention the pharmaceutically acceptable salt of compounds 1 (c-d), 1 (f-i), 1 (o-p), 1(u-z) is selected from a group consisting of selected from a group consisting of hydrochloride and tartrate salts.

In yet another embodiment of the present invention the % aggregation of compounds by collagen induced platelet aggregation in human platelets (in vitro) varies from 03.00±3.00 to 86.00±3.41% at 30 μM concentration. The compound 1d was the most potent among these groups exhibiting a percentage inhibition of aggregation of 86.000±3.41 induced by collagen.

In still another embodiment of the present invention, the Compounds 1d, 1g, 1h, 1o, 1u, 1v and 1w exhibited highly promising anti-platelet efficacy inhibited collagen, in vitro varies from 57.00±11.00 to 86.00±3.41% and Compound 1d was the most potent among these groups and exhibited a percent inhibition of aggregation of 86.00±3.41, induced by collagen.

In yet another embodiment of the present invention, the compounds 1d, 1g, 1h, 1u, 1v and 1w exhibited dose dependent anti-platelet efficacy through dual mechanism inhibited both collagen inhibited both collagen as well as U46619 (thromboxane receptor agonist) induced platelet aggregation and varies from 52±03 to 85±03.

In still another embodiment of the present invention, Compound 1d was evaluated for its antithrombotic efficacy in ferric chloride induced arterial thrombosis model in mice and after 4 hr of its oral administration, prolonged the time to occlusion of carotid artery by 2.2 fold (control, 9.5±0.4 min vs 1d, 19.2±0.9 min), while the standard drug Clopidogrel increased the TTO upto 23±0.9 min. Therefore, the efficacy elicited in this model substantiates the anti-thrombotic potential of this compound.

In yet another embodiment of the present invention, the action of compound 1d is platelet specific, since its presence did not alter the coagulability of blood as assessed by TT, PT and aPTT in human plasma.

### BRIEF DESCRIPTION OF THE DRAWINGS

Scheme 1: Coupling reactions involving N-substituted pyroglutamic acid

Scheme 2: Coupling reactions involving N-protected hydrophobic amino acids.

Scheme 3: Modifications of bispidine ring.

Table 1. In vivo (% protection; inducer, collagen plus epinephrine) and in vitro (% inhibition of aggregation; inducer, collagen) activity of bispidine derivatives of N-substituted pyroglutamic acid, 1(a-w).

Table 2. In vivo (% protection; inducer, collagen plus epinephrine) and in vitro (% inhibition of aggregation; inducer, collagen) activity of bispidine derivatives of N-protected amino acids, 1(x-z).

FIG. 1: Effect of compound 1d against (a) collagen induced aggregation in human platelets (in vitro), (b) U46619 induced platelet aggregation, (c) ADP, TRAP, Ristocetin, CRP-XL and arachidonic acid induced platelet aggregation in human, platelets. Results are expressed as Mean±SEM (n=3). Bars in graph (a) and (b) represents percent inhibition (Mean±SEM) offered by compound 1 d against human platelet aggregation induced by collagen and U46619 respectively. Bars in graph (c) represents percent platelet aggregation (Mean±SEM) induced by ADP, TRAP, Ristocetin, CRP-XL and arachidonic acid in presence of vehicle/compound 1d.

FIG. 2: Effect of compound 1d on Tail bleeding time in mice after (a) 1 hr (b) 4 hr of oral administration. Results are expressed as Mean±SEM (n=5, 10 animals/group/experiment).

FIG. 3: Effect of compound 1d on total time to occlusion (TTO) in ferric chloride induced arterial thrombosis in mice (n=6).

### ABBREVIATIONS

ADP: Adenosine Diphosphate, TxA2: Thromboxane A2, LiHMDS: Lithium bis(trimethylsilyl)amide, Boc: tert-butyloxycarbonyl, TFA: Triflouroacetic acid, DCC: Dicyclohexyldicarbodiimide, DCM: Dichloromethane, HOBt: 1-Hydroxybenzotriazole, TEA: Triethylamine, TDW: triple distilled water, CPD: citrate-phosphate-dextrose, PRP: Platelet-rich plasma, ACD: Acid Citrate Dextrose, HEPES: 4-(2-hydroxyethyl)-1-piperazineethanesulfonic acid, EGTA: ethylene glycol tetraacetic acid, BSA: bovine serum albumin, TRAP: thrombin receptor activating peptide; TTO: total time to occlusion; CRP: collagen-related peptide; TT: thrombin time; PT: prothrombin time; aPTT: Activated Partial Thromboplastin Time; COX: cyclooxygenase; DIPEA: N,N-Diisopropylethylamine; PyBOP: benzotriazol-1-yl-oxytripyrrolidinophosphonium hexafluorophosphate

### DETAILED DESCRIPTION OF THE INVENTION

The present invention provides N-substituted pyroglutamic acids and substituted/protected amino acids condensed with substituted bispidines and a process for the preparation of the said compounds of general formula 1, respectively, useful in antithrombotic activity.

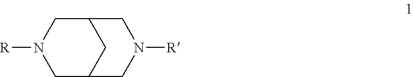

Wherein, R′ is;

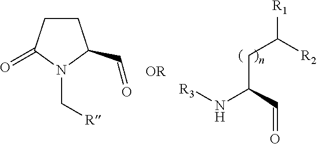

Wherein R is selected from alkyl, acyl, tosyl, tert-butyloxycarbonyl or substituted araalkyl groups; R″ is selected preferably from halogen, cyano, lower alkyl, aryl, substituted aryl and tosyl groups; R₁ is selected from hydrogen and lower alkyl groups; R₂ is selected from lower alkyl and aryl groups; R₃ is selected from tert-butyloxycarbonyl and bezyloxycarbonyl groups; n=0,1.

The compounds synthesized were tested for antiplatelet activities. A number of these compounds showed protection against collagen-epinephrine induced pulmonary thromboembolism in mice, in vivo and Inhibition of collagen as well as U46619 induced platelet aggregation (in vitro) in human platelets.

Accordingly, the present invention provides a process for the preparation of general formula 1, wherein the process steps comprising of intermediates 2, 3, 4 and 5 and were prepared by the reported procedures,

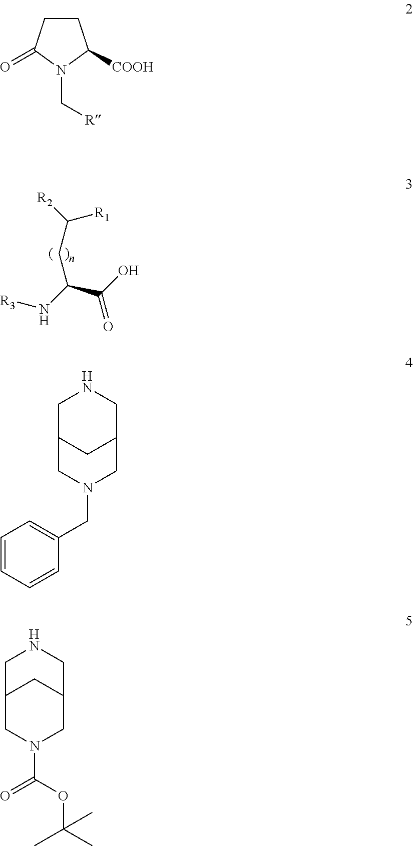

R″ is selected preferably from halogen, cyano, lower alkyl, aryl, substituted aryl and tosyl groups; R₁ is selected from hydrogen and lower alkyl groups; R₂ is selected from lower alkyl and aryl groups; R₃ is selected from tert-butyloxycarbonyl and bezyloxycarbonyl groups; n=0,1.
Further process steps comprising;
i) Reacting compound of formula 2 with compound of formula 4 or 5 in an aprotic solvent selected form a group consisting of dichloromethane, tetrahydrofuran, dioxane in presence of a coupling reagent selected from the group consisting of dicyclohexylcarbodiimide, benzotriazole-1-yl-oxy-tris-pyrrolidino-phosphonium hexafluorophophate, OR an activating agent 1-hydroxy benzotrizole or isobutyl chloroformate at −20° C., followed by stirring at 30° C. for a period of 3 hrs followed by purification using chromatography (silica gel 60-120 mesh) to produce compound of formula 1 (Scheme1).
   In an embodiment of the invention wherein the compound of formula 2 is reacted with oxalyl chloride at 0° C. to obtain the acid chloride followed by reaction with compound of formula 4 or 5 in presence of triethylamine (TEA) in dichloromethane at 25° C. for a period ranging from 2h to 3 h to obtain the compound of formula 1 (Scheme1).
   In another embodiment of the invention wherein the compound of formula 2 is reacted with compound of formula 4 or 5 in presence of a coupling reagent dicyclohexylcarbodiimide (DCC) and 1-hydroxybenzotrizole (HOBt) in dichloromethane at −5° C. for a period of 3h to get compound of formula 1 (Scheme1).
   In a further embodiment of the invention wherein the compound of formula 2 is reacted with compound of formula 4 or 5 in the presence of diisopropylethylamine (DIPEA), and benzotriazole-1-yl-oxy-tris-pyrrolidino-phosphoniumhexafluorophophate, (PyBOP), in dichloromethane at 0° C. for 3h followed by stirring at 0° C. for 1h and then at 27° C. for 2h to obtain compound of formula 1 (Scheme1).
   In still another embodiment of the invention wherein the compound of formula 2 is reacted with compound of formula 4 or 5 in presence of TEA, and isobutyl chlorormate, in THF at −20° C., for 2h followed by stirring at 0° C. for 1h and then at 25° C. for 2h to obtain compound of formula 1 (Scheme1).

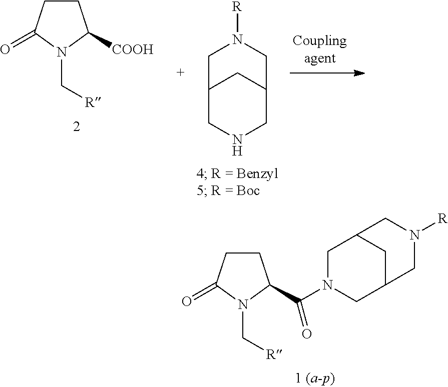

wherein R is selected from alkyl, tert-butyloxycarbonyl or substituted araalkyl groups; R″ is selected from halogen, cyano, lower alkyl, alkoxy substituted aryl groups.
ii) Synthesis of amino acid derivatives of substituted bispidines, 1(x-z) by reacting compound of formula 3 with compound of formula 4 or 5 as illustrated as in the case of Scheme-1 to obtain compound of general formula. (Scheme-2)

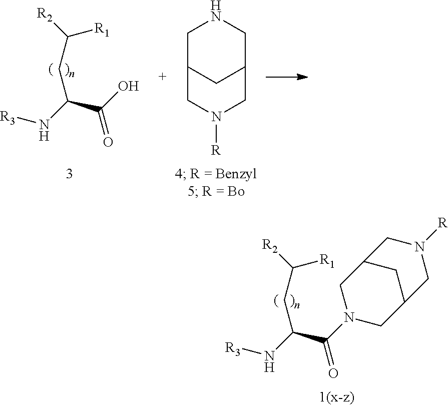

wherein R is selected from alkyl, tert-butyloxycarbonyl or substituted araalkyl groups; R₁ is selected from hydrogen and lower alkyl groups; R₂ is selected from lower alkyl and aryl groups; R₃ is selected from tert-butyloxycarbonyl and bezyloxycarbonyl groups; n=0,1.
iii) Synthesis of 3-(N)-acyl, sulfonyl and substituted benzyl analogs of the compound 1 by modifying the bispidine portion to obtain compounds of general formula 1(q-w). (Scheme 3).

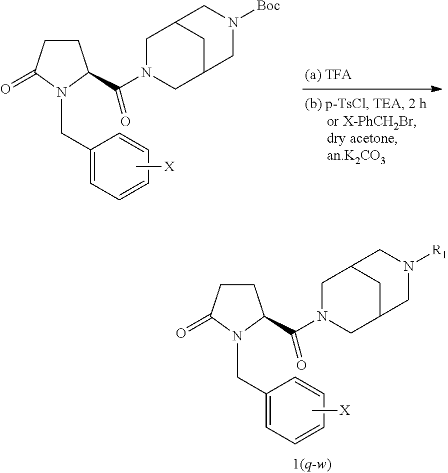

wherein R₁ is selected from substituted acyl, tosyl, groups or substituted benzyl groups; X is selected from halogen, cyano, lower alkyl or alkoxy groups.

### EXAMPLES

### The Following Examples are Given by Way of Illustrating the Present Invention and should not be Construed to Limit the Scope of the Present Invention

### Example 1

### General Synthesis of (2S)—N-arylalkyl pyroglutamic acid, (2)

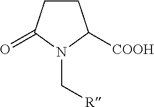

A solution of Methyl pyroglutamate, 7 (2.0 gm, 1 eq, 13.9 mmol) and THF (100 ml, freshly distilled over benzophenone ketyl radical) was taken in a three necked RBF fitted with rubber septa, N₂ inlet and cooled to −20° C. LiHMDS (14 ml, 1.2 eq, 16.7 mmol) was added through a syringe to that solution and allowed to stir for 1 h. Benzylbromide (2.85 g, 1.1 eq, 15.4 mmol) was added and stirring was continued for 4h from 0° C. to 25° C. The reaction was quenched by addition of 1N HCl (10 ml) and extracted with ethyl acetate (3×25 ml). The organic layer was washed with brine (2×25 ml), dried over Na₂SO₄ and concentrated under reduced pressure to give an oily ester, 8. This ester was then dissolved in methanol (10 ml) and cooled to 0° C. 20% sodium carbonate solution was then added to the reaction mixture portion wise. The reaction mixture was then stirred 25° C. for 5 hours. Methanol was then distilled off and the reduced reaction mixture was then extracted with ether (1×25 ml). The mixture was acidified with conc.HCl and extracted with ethyl acetate (3×30 ml). The organic layer was dried and concentrated.

Yield: 40%; M.P.: 86-88° C.; [α]_{D}^{27° C.}: +33.96 (c=0.10; Methanol); IR (Neat): 3758, 3452, 2962, 1969, 1663, 1453, 1422, 1281, 1024, 801 cm⁻¹; ¹H NMR (CDCl₃, 200 MHz): □□2.05-2.18 (m, 1H, 3-Hₐ); 2.20-2.27 (m, 1H, 3-H_{b}); 2.32 (s, 3H, —CH₃); 2.50-2.60 (m, 2H, 4-H); 3.88-3.92 (d, 1H, —NCHPh); 4.02-4.04 (m, 1H, 2-H); 5.09-5.17 (d, 1H, —NCHPh); 7.12 (s, 5H, Ph-H); ¹³C NMR (CDCl₃, 200 MHz): 14.57, 21.52, 23.26, 30.10, 45.83, 59.01, 61.00, 128.95, 129.92, 132.62, 138.11, 174.74, 176.90; FAB MS (m/z): 234 (M+H)⁺

### Example 2

### 4-Oxo-piperidine-1-carboxylic acid tert-butyl ester, (10)

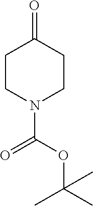

A solution of piperidin-4-one, 9 (5.0g, 1 eq, 0.032 mol) in THF was cooled to 0° C. and 20% aqueous solution of sodium bicarbonate (100 ml.) was added portion wise to the stirring reaction mixture. A solution of di-tert-butyl-dicarbonate (6.984g, 1 eq, 0.032 mol) in THF was added drop wise to the stirring reaction mixture at 0° C. and continued to stir at 25° C. for 3 to 4 hours. The reaction mixture was extracted with ethyl acetate. The organic layer was washed with brine The combined organics were dried with anhydrous Na₂SO₄, concentrated to obtain pale yellow oily liquid which turned to pale white solid (6.4g). The residue was purified by column chromatography on silica gel (n-hexane/ethyl acetate=4/1) to obtain pure compound (5.671 g).

Yield: 87.44%; MP: 63° C.; IR (KBr): 2979.1, 2938.9, 2868.1, 1686.1, 1424.6, 1366.2, 1318.1, 1242.3, 1166.7, 1115.1 cm⁻¹; ¹H NMR (300 MHz, CDCl₃, ppm): δ 3.73 (t, 2H, CH₂NC(O)); 2.45 (t, 2H, CH₂C(O)); 1.50 (s, 9H, CMe₃); ¹³C NMR (50 MHz, CDCl₃, ppm): δ 207 (C═O), 154 (Boc C═O), 80.5 (CMe₃), 43.0 (CH₂NC(O)), 41.1 (CH₂), 28.3 (CH₃).

### Example 3

### 7-Benzyl-9-oxo-3,7-diaza-bicyclo[3.3.1]nonane-3-carboxylic acid tert-butyl ester, (11)

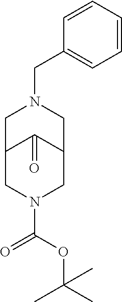 [SMILES: CC(C)(C)OC(=O)N1CC2CN(Cc3ccccc3)CC(C1)C2=O]

A solution of 4-Oxo-piperidine-1-carboxylic acid tert-butyl ester, 10 (4.0g, 1 eq, 0.020 mol), acetic acid (1.145 ml, 1 eq, 0.020 mol) and benzylamine (2.229 ml, 1.1 eq, 0.0204 mol) in methanol was added drop wise to the stirring suspension of paraformaldehyde (1.2g, 2 eq, 0.04 mol) in methanol (40 ml) at 65° C. and allowed to heat at reflux for 1 hr. After 1 hr., it was allowed to cool and a second portion of paraformaldehyde (1.2g, 2 eq, 0.04 mol) was added and reaction mixture was heated at reflux for 4 hrs this time. After being cooled to 25° C., the solvent was evaporated under reduced pressure. The residue was dissolved in diethyl ether and washed with 1M KOH. The organic layer was washed with brine. The combined organics were dried with anhydrous Na₂SO₄ concentrated to obtain pale yellow sticky material (6.426g). The crude product was purified by column chromatography on silica gel (n-hexane/ethyl acetate=9/1) to obtain pure product (3.678 g).

Yield=55.45%; MP: 78° C.; IR (KBr): 3015.2, 2929.9, 2864.8, 2806.0, 1730.8, 1688.4, 1424.7, 1232.1, 1168.0 cm⁻¹; ¹H NMR (300 MHz, CDCl₃, ppm): δ 7.23-7.26 (m, 5H, Ph-H); 4.61-4.57 (brd, J=12 Hz, 1H, C(O)NCH); 4.45-4.41 (brd, J=12 Hz, 1H, C(O)NCH); 3.54-3.52 (d, J=6 Hz, 2H, CH₂Ph); 3.40-3.32 (m, 2H, 2×C(O)NCH); 3.32-3.19 (m, 2H, 2×NCH); 2.75-2.66 (d, 1H, 2×NCH), 2.46-2.44 (m, 2H, 2×CH), 1.55 (s, 9H, CMe₃); ¹³C NMR (50 MHz, CDCl₃, ppm) δ213.56 (bridge C═O), 154.78 (Boc C═O), 137.45 (ipso Ph), 128.77 (Ph), 128.33 (Ph), 127.26 (Ph), 80.09 (CMe₃), 61.84 (CH₂Ph), 50.49 (C(O)NCH), 47.59 (2×CH), 28.59 (CH₃).

### Example 4

### 7-Benzyl-3,7-diaza-bicyclo[3.3.1]nonane-3-carboxylic acid tert-butyl ester, (12)

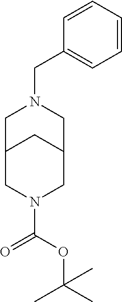 [SMILES: CC(C)(C)OC(=O)N1CC2CC(CN(Cc3ccccc3)C2)C1]

To a mixture of 4-Oxo-piperidine-1-carboxylic acid tert-butyl ester (2g, 1 eq, 6.1 mmol) hydrazine monohydrate (0.33 g, 1.1 eq, 6.6 mmol) and diethylene glycol (27.83 ml) was added. At 60° C., powdered KOH (2.504g) was added to the reaction mixture and again heated at 160° C. for 8 hrs. Then the mixture was cooled and water (40 ml) was added and allowed to stir. The reaction mixture was extracted with dichloromethane and combined organics were dried with anhydrous Na₂SO₄ concentrated to obtain oily residue (2.331g). The crude product was purified by column chromatography on silica gel (n-hexane/ethyl acetate=9/1) to obtain pure product.

Yield=57.54%; IR (Neat): 3016.3, 2922.7, 1672.4, 1427.3, 1217.4, 1174.6 cm⁻¹; ¹H NMR (300 MHz, CDCl₃, ppm) δ7.36-7.23 (m, 5H, Ph-H); 4.19-4.13 (br d, J=18 Hz, 1H, CONCH); 4.03-3.99 (br d, J=12 Hz, 1H, CONCH); 3.48-3.44 (d, J=12 Hz, 1H, CH_{2A}Ph); 3.34-3.30 (d, J=12 Hz, 1H, CH_{2B}Ph); 3.13-3.09 (m, 2H, 2×CONH); 3.03-2.98 (br d, J=15 Hz, 1H, NCH); 2.92-2.89 (br d, J=9 Hz, 1H, NCH); 2.25-2.17 (m, 2H, 2×NCH); 1.89 (br s, 1H, CH); 1.81 (br s, 1H, CH); 1.68 (m, 2H, bridge CH₂); 1.54 (s, 9H, CMe₃); ¹³C NMR (50 MHz, CDCl₃, ppm) δ155.15 (C═O), 128.68 (Ph), 128.10 (Ph), 126.68 (Ph), 78.83 (CMe₃), 63.49 (CH₂Ph), 58.77 (NCH₂), 48.43 (CONCH₂), 47.65 (CONCH₂), 37.63 (bridge-CH₂), 31.10 (2×CH), 28.72 (CH₃); MS (ESI): 317.3 (M+H)⁺

### Example 5

### 3-Benzyl-3,7-diaza-bicyclo[3.3.1]nonane, (4)

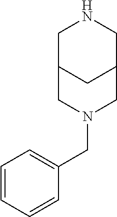 [SMILES: c1ccc(CN2CC3CNCC(C3)C2)cc1]

Trifluoro acetic acid (TFA) (2.25 ml, 5 eq, 0.03 mol) was injected to the stirring suspension of compound 12 (2.0g, 1 eq, 0.006 mol) in DCM at 0° C. and allowed to stir at 25° C. (25-35° C.). Then reaction mixture was made alkaline by adding 20% aq. solution of Na₂CO₃ and resulting mixture was extracted with dichloromethane (3×50 ml) and organics were washed with brine. The combined organics were dried with anhydrous Sodium sulphate and concentrated to obtain yellow oily liquid (1.641g).

Yield: 90%; IR(Neat): 3451.4, 2924.6, 1610.0, 1450.4 cm⁻¹; ¹H NMR (300 MHz, CDCl₃, ppm): δ7.37-7.27 (m, 5H, Ph-H); 3.49-3.41 (m, 4H, CH₂Ph, 2×NCH); 3.31-3.27 (d, J=12 Hz, 2H, 2×NCH); 3.19-3.11 (m, 2H, 2×NCH); 2.49-2.45 (d, J=12 Hz, 2H, 2×NCH); 2.12-2.07 (m, 2H, 2×CH); 1.93-1.89 (d, J=12 Hz, 1H, bridge CH); 1.79-1.75 (d, J=12 Hz, 1H, bridge CH); MS (ESI): m/z=217 (M+H)⁺

### Example 6

### 3,7-Diaza-bicyclo[3.3.1]nonane-3-carboxylic acid tert-butyl ester, (5)

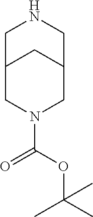 [SMILES: CC(C)(C)OC(=O)N1CC2CNCC(C2)C1]

Palladium hydroxide (0.5g), (Pearlman's catalyst), was added portion wise to a suspension of compound 12 (1.102g, 0.0367 mol) in methanol (25 ml) in steel parr. The reaction mixture was hydrogenated at 55° C. and 150 psi for about 17 hrs. Then it was allowed to cool and filtered over sintered funnel with the aid of vacuum and concentrated to get pale yellow solid.

Yield=93.33%; MP: 75° C.; IR (KBr): 2979.2, 2919.7, 2858.5, 1679.6, 1402.4, 1240.4, 1172.31131.9 cm⁻¹; 1H NMR (300 MHz, CDCl₃, ppm): δ4.13-4.09 (brd, J=12 Hz, 2H, 2×CONCH); 3.14-3.10 (m, 3H, 2×CONCH, NCH); 3.01-2.96 (brd, J=15 Hz, 1H, NCH); 2.25 (s, 2H, 2×NH); 1.92-1.88 (d, J=12 Hz, 1H, CH); 1.80-1.76 (d, J=12 Hz, 1H, CH); 1.67 (s, bridge CH₂); 1.48 (s, 9H, CMe₃); ¹³C NMR (50 MHz, CDCl₃, ppm): δ155.49 (C═O), 79.78 (CMe₃), 51.50 (NCH₂), 48.94 (C(O)NCH₂), 31.39 (CH), 28.52 (CMe₃), 28.15 (CMe₃); MS (ESI): 227.1481 (M+H)⁺

### Example 7

### 7-(1-Benzyl-5-oxo-pyrrolidine-2-carbonyl)-3,7-diaza-bicyclo[3.3.1]nonane-3-carboxylic acid tert-butyl ester, (1a)

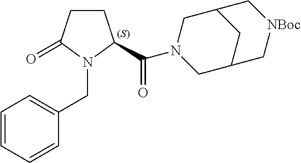

DCC (308 mg, 1.2 eq, 1.495 mmol) dissolved in DCM (5 ml) was added to the stirring reaction mixture containing N-benzyl pyroglutamic acid, 3 (273 mg, 1 eq, 1.25 mmol) and HOBt (252.58 mg, 1.5 eq, 1.86 mmol) dissolved in dry DCM (10 ml) at 0° C. and continued to stir for 15 minutes at same temperature. Then N-Boc bispidine, 5 (281.82 mg, 1 eq, 1.25 mmol) dissolved in dry DCM (5 ml) was added drop wise to the stirring reaction mixture and continued to stir for about 2-3 hrs. The reaction mixture was then brought to 25° C. and concentrated. The concentrated mass was then dissolved in diethyl ether and washed successively with dilute citric acid (1×20 ml), dilute NaHCO₃ (1×20 ml), brine and then extracted with ethyl acetate (3×20 ml). The combined organics were dried with anhydrous Na₂SO₄ and concentrated to obtain sticky oily product (534 mg).

Yield=59.17%; [α]_{D}^{27° C.}=−15.1890 (Methanol, c=0.3160); IR (Neat): 3017.0, 2366.7, 2337.8, 1678.9, 1432.0, 1217.9 cm⁻¹; ¹H NMR (300 MHz, CDCl₃, ppm): δ 7.36-7.14 (m, 5H, Ph-H); 5.15-5.10 (brd, J=15 Hz, 1H, PhCH_{A}); 4.59-4.54 (brd, J=12 Hz, 1H, NC_{2′}H_{A}); 4.09-4.06 (m, 2H, NC₂H, PhCH_{B}); 3.81-3.76 (br d, J=15 Hz, 1H, NC_{8′}H_{A}); 3.51-3.47 (d, J=12 Hz, 1H, NC_{2′}H_{B}); 3.08-2.86 (m, 4H, PhCH_{B′}, NC_{8′}H_{B}, NC_{4′}H_{A}, C_{6′}H₂); 2.51-2.40 (m, 2H, C₄H_{A}, C_{4′}H_{B}); 2.26-2.16 (m, 1H, C₄H_{B}); 2.16-1.89 (m, 4H, C₃H₂, C_{3′}H, C_{7′}H); 1.70 (s, 2H, C_{9′}H₂); 1.41 (s, 9H, CMe₃); ¹³C NMR (50 MHz, CDCl₃, ppm): δ 176.16 (COOH), 174.85 (C═O), 135.40 (Ph), 128.84 (Ph), 128.53 (Ph), 127.95 (Ph), 49.48 (NCH), 49.00 (NCH₂), 45.36 (NC_{2′}, NC_{8′}), 29.99 (C_{9′}), 34.49 (bridge CH₂), 28.35 (C_{3′}), 27.70 (C_{7′}), 27.32 (CMe₃), 22.88 (CH₂); MS (ESI): m/z=427.9 (M⁺)

### Example 8

### 7-[1-(2-Bromo-benzyl)-5-oxo-pyrrolidine-2-carbonyl]-3,7-diaza-bicyclo[3.3.1]nonane-3-carboxylic acid tert-butyl ester, (1b)

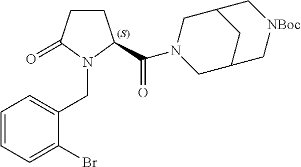

The compound was prepared from N-(2-bromobenzylpyroglutamic acid using DCC (249.08 mg, 1.2 eq, 1.207 mmol) containing and HOBt (203.91 mg, 1.5 eq, 1.509 mmol) dissolved in dry DCM (10 ml) followed by the addition of N-Boc bispidine, 5 (227.51 mg, 1 eq, 1.006 mmol) dissolved in dry DCM.

Yield: 49.64%; MP: 138° C.; [α]_{D}^{27° C.}: 3.7608 (Methanol, c=0.2180); IR (KBr): 3458.8, 2927.9, 1679.6, 1434.4, 1241.5, 1172.5, 1134.3 cm⁻¹; ¹H NMR (300 MHz, CDCl₃, ppm): δ 7.5-7.1 (m, 4H, Ph-H); 5.1-5.0 (d, 1H, PhCH_{A}); 4.5 (d, 1H, NC_{2′}H_{A}); 4.1-4.0 (m, 2H, NC₂H, PhCH_{B}); 3.5 (d, 1H, NC_{8′}H_{B}); 3.1-2.8 (m, 4H, NC_{2′}H_{B}, NC_{8′}H_{B}, NC_{4′}H, NC_{6′}H_{A}); 2.4-2.3 (m, 3H, C₄H_{A}, NC_{4′}H, NC_{6′}H_{B}); 2.2-2.0 (m, 2H, C₄H_{B}, C₃H_{A}); 1.9 (m, 1H, C₃H_{B}); 1.8 (m, 2H, C_{3′}H, C_{7′}H); 1.7 (s, 2H, C_{9′}H₂); 1.4 (s, 9H, CMe₃); ¹³C NMR (50 MHz, CDCl₃, ppm): δ135.84 (Ph), 132.75 (Ph), 131.39 (Ph), 129.38 (Ph), 127.76 (Ph), 124.13 (Ph), 79.73 (CMe₃), 56.85 (CCH₃), 49.59 (COONCH), 46.51 (NCH), 45.30 (OCONCH), 34.64 (bridge CH₂), 28.32 (CH), 28.13 (CH), 27.70 (CMe₃), 27.29 (CMe₃), 22.82 (CMe₃); MS(ESI): m/z: 528.0 (M+Na)⁺

### Example 9

### 1-Benzyl-5-(7-benzyl-3,7-diaza-bicyclo[3.3.1]nonane-3-carbonyl)-pyrrolidin-2-one, (1c)

Step 1: N-benzylpyroglutamic acid (329 mg, 1 eq, 1.369 mmol) dissolved in dry DCM (15 ml) was cooled to 0° C. and oxalyl chloride (0.191 ml, 1.5 eq, 2.053 mmol) was added drop wise to the stirring reaction mixture at same temperature and allowed to stir overnight at 25° C. The reaction mixture was concentrated to evaporate DCM.

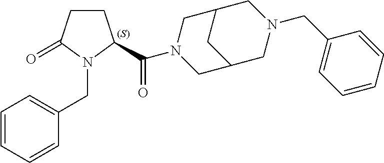

Step 2: N-benzyl bispidine (349.18 mg, 1 eq 1.617 mmol) dissolved in dry DCM (10 ml) was cooled to 0° C. and triethyl amine (0.471 ml, 2.3 eq, 3.3803 mmol) was added drop wise to the stirring reaction mixture. Then concentrated mass from step-1 dissolved in dry DCM was added drop wise at same temperature and continued to stir for 2-3 hrs.

Yield=30.32%; [α]_{D}^{27° C.}=+3.44 (Methanol, c=0.2120); MP: 144° C.; IR (KBr): 3424.2, 3010.2, 2924.5, 1680.5, 1642.6, 1449.8, 1218.9 cm⁻¹; ¹H NMR (300 MHz, CDCl₃, ppm): δ 7.29-7.22 (m, 10H, 2×Ph); 5.23-5.18 (m, 1H, PhCH_{A}); 4.60-4.45 (m, 1H, NC_{2′}H_{A}); 4.17-4.15 (m, 1H, NC₂H); 3.85-3.79 (m, 1H, PhCH_{B}); 3.50-3.45 (m, 2H, PhCH_{A′}, NC_{′}H_{A}); 3.26-3.22 (m, 1H, PhCH_{B′}); 3.08-2.99 (m, 3H, NC_{2′}H_{B}, NC_{8′}H_{B}, NC_{4′}H_{A}); 2.85 (m, 1H, NC_{6′}H_{A}); 2.57 (m, 1H, C₄H_{A}); 2.38-2.33 (m, 1H, C_{4′}H_{B}, NC_{6′}H_{B}); 2.10-2.06 (m, 2H, C₄H_{B}, C₃H_{A}); 1.97 (m, 1H, C₃H_{B}); 1.88 (m, 2H, C_{3′}H, C_{7′}H); 1.70 (s, 2H, C_{9′}H₂); ¹³C NMR (50 MHz, CDCl₃, ppm): δ175.67 (C═O), 168.41 (C═O), 128.64 (Ph), 128.54 (Ph), 128.35 (Ph), 128.07 (Ph), 127.58 (Ph), 127.05 (Ph), 63.52 (NCH₂Ph), 59.35 (C_{6′}), 58.38 (C_{4′}), 56.54 (NCH₂), 46.44 ( ), 45.40 (NC_{2′}), 31.04 (bridge CH₂), 29.98 (C₄), 29.19 (C_{3′}), 28.54 (C_{7′}), 21.56 (CH₂); MS (ESI): m/z=418.2 (M+H)⁺

### Example 10

### 5-(7-Benzyl-3,7-diaza-bicyclo[3.3.1]nonane-3-carbonyl)-1-(2-bromo-benzyl)-pyrrolidin-2-one (1d)

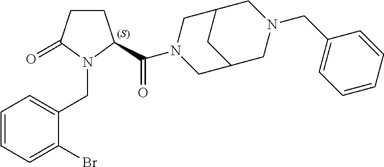

Step 1: N-2-bromobenzylpyroglutamic acid (300 mg, 1 eq, 1.006 mmol) dissolved in dry DCM (10 ml) was cooled to 0° C. and oxalyl chloride (0.127 ml, 1.5 eq, 1.509 mmol) was added drop wise to the stirring reaction mixture at same temperature and allowed to stir overnight at 25° C. (25-35° C.).

Step 2: N-benzyl bispidine dissolved (239.02 mg, 1 eq 1.1066 mmol) in dry DCM (10 ml) was cooled to 0° C. and triethylamine (0.322 ml, 2.3 eq 2.314 mmol) was added drop wise to the stirring reaction mixture. Then concentrated mass from step-1 dissolved in dry DCM was added drop wise at same temperature and continued to stir for 2-3 hrs.

Yield=54.04%; MP: 131° C.; [α]_{D}^{27° C.}: +25.5130 (Methanol, c=0.2040); IR (KBr): 3464.8, 3354.4, 2911.5, 2802.9, 1690.2, 1638.9, 1442.4, 1342.4, 1285.8, 1254.6 cm⁻¹; ¹H NMR (300 MHz, CDCl₃, ppm): δ 7.29-7.22 (m, 9H, 2×Ph); 5.14-5.09 (m, 1H, PhCH_{A}); 4.57-4.53 (d, J=12 Hz, 1H, NC_{2′}H_{A}); 4.21-4.09 (m, 2H, NC₂H, PhCH_{B}); 3.50-3.43 (m, 2H, PhCH_{A}, NC_{8′}H_{A}); 3.25-3.16 (m, 2H, PhCH_{B′}, NC_{2′}H_{B}); 3.0-2.84 (m, 3H, NC_{8′}H_{B}, NC_{4′}H_{A}, C_{6′}H_{A}); 2.61-2.49 (m, 1H, C₄H_{A}); 2.35-2.27 (m, 2H, NC_{4′}H_{B}, C_{6′}H_{B}); 2.05-1.93 (m, 1H, C₃H_{B}); 1.89 (m, 2H, C_{3′}H, C_{7′}H); 1.68 (s, 2H, C_{9′}H₂);

¹³C NMR (50 MHz, CDCl₃, ppm): δ 175.72 (C═O), 168.34 (C═O), 137.90 (Ph), 136.149 (Ph), 132.84 (Ph), 128.61 (Ph), 128.33 (Ph), 127.77 (Ph), 127.01 (Ph), 124.43 (Ph), 115.35 (Ph), 63.83 (NCH₂Ph), 59.73 (NCH₂), 58.56 (NCH), 56.97 (CH₂Ph), 49.29 (NCH₂), 45.67 (COCH₂), 34.64 (bridge CH₂), 31.33 (CH), 29.20 (CH), 21.96 (CH₂); MS (ESI): m/z=496.2 (M+H)⁺

### Example 11

### 7-[1-(4-Methyl-benzyl)-5-oxo-pyrrolidine-2-carbonyl]-3,7-diaza-bicyclo[3.3.1]nonane-3-carboxylic acid tert-butyl ester, (1e)

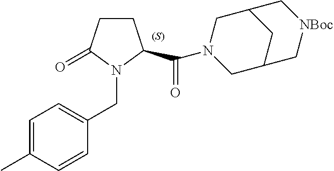

Compound 5 (305.53 mg, 1 eq, 1.350 mmol) dissolved in dry DCM (10 ml) was added to N-(4-methylbenzyl)pyroglutamic acid (314 mg, 1 eq, 1.350 mmol) dissolved in dry DCM (5 ml). Then DIPEA (0.470 ml, 2 eq, 2.76 mmol) was added drop wise to the stirring reaction mixture at 0° C. under nitrogen atmosphere. Then PyBOP (702.52 mg, 1 eq, 1.350 mmol) dissolved in dry DCM was added drop wise to the stirring reaction mixture at same temperature and continued to stir for about 3 hrs. The reaction mixture was washed successively with 20% citric acid (1×20 ml), 20% NaHCO₃ (1×20 ml) and brine. The combined organics were dried with anhydrous Na₂SO₄ and concentrated to get the sticky oily product. Then it was purified by column chromatography on silica gel to obtain pure product.

Yield: 50.91%; [α]_{D}^{27° C.}: −16.7970 (Methanol, c=0.0980); IR (Neat): 3412.3, 2925.2, 1667.9, 1423.3, 1364.3, 1245.4, 1172.5, 1135.0 cm⁻¹; ¹H NMR (300 MHz, CDCl₃, ppm): δ 7.28-7.04 (m, 4H, Ph); 5.13-5.08 (d, J=15 Hz, PhCH_{A}); 4.61-4.56 (m, 1H, NC_{2′}H_{A}); 4.15-4.07 (m, 1H, NC₂H); 3.76-3.71 (d, J=15 Hz, 1H, PhCH_{B}); 3.54-3.50 (d, J=12 Hz, 1H, NC_{8′}H_{A}); 3.04-2.94 (m, 4H, NC_{2′}H_{B}, NC_{8′}H_{B}, NC_{4′}H_{A}, NC_{6′}H_{A}); 2.34 (s, 1H, CH₃); 2.22-2.00 (m, 6H, C₄H_{A}, C_{6′}H_{B}, C_{4′}H_{B}, C₄H_{B}, C₃H₂); 1.91-1.90 (m, 2H, C_{3′}H, C_{7′}H); 1.80 (s, 2H, C_{9′}H₂); 1.42 (s, 9H, CMe₃); ¹³C NMR (50 MHz, CDCl₃, ppm): δ 175.68 (C═O), 168.33 (C═O), 137.29 (Ph), 129.28 (Ph), 128.58 (Ph), 49.52 (NCH₂), 45.03 (NCH₂), 34.64 (bridge CH₂), 30.04 (CH₂), 28.35 (CMe₃), 27.74 (CH₃), 27.35 (CH₃), 22.81 (CH₃), 21.08 (PhCMe₃); MS (ESI): m/z=441.9 (M⁺)

### Example 12

### 5-(7-Benzyl-3,7-diaza-bicyclo[3.3.1]nonane-3-carbonyl)-1-(4-methyl-benzyl)-pyrrolidin-2-one (1f)

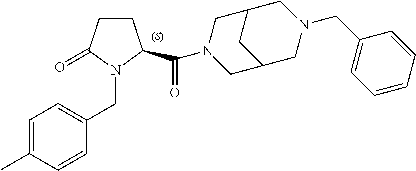

DCC (335.69 mg, 1 eq, 1.36 mmol) dissolved in dry DCM (5 ml) was added to the stirring reaction mixture containing N-(4-methyl benzyl) pyroglutamic acid (316.30 mg, 1 eq, 1.356 mmol) and HOBt (274.85 mg, 1.2 eq 1.627 mmol) dissolved in dry DCM (10 ml) at 0° C. and continued to stir for 15 minutes at same temperature. Then N-benzyl bispidine (293 mg, 1 eq, 1.356 mmol) dissolved in dry DCM (5 ml) was added drop wise to the stirring reaction mixture and continued to stir for about 2-3 hrs. The reaction mixture was then brought to 25° C. and concentrated. The concentrated mass was then dissolved in diethyl ether and washed successively with 20% citric acid (1×20 ml), 20% NaHCO₃ (1×20 ml), brine and then extracted with ethyl acetate (3×20 ml). The combined organics were dried with anhydrous Na₂SO₄ and concentrated to obtain sticky oily product which get solidified later. Then it was purified by column chromatography on silica gel (DCM: Methanol=7:3) to obtain pure product.

Yield=59.13%; MP: 133° C.; [α]_{D}^{27° C.}: +0.9200 (Methanol, c=0.1260); IR (KBr): 3445.7, 2362.3, 1637.4, 1466.5, 1219.1 cm⁻¹; ¹H NMR (300 MHz, CDCl₃, ppm): δ 7.30-7.07 (m, 9H, 2×Ph); 5.18-5.14 (br d, J=12 Hz, 1H, PhCH_{A}); 4.59-4.45 (m, 1H, NC_{2′}H_{A}); 4.17-4.15 (m, 1H, NC₂H); 3.79-3.74 (m, 1H, PhCH_{B}); 3.51-3.47 (m, 2H, PhCH_{A′}, NC_{8′}H_{A}); 3.27-3.10 (m, 1H, PhCH_{B′}); 3.02-2.86 (m, 4H, NC_{2′}H_{B}, NC_{8′}H_{B}, NC_{4′}H_{A}, NC_{6′}H_{A}), 2.55 (m, 2H, C₄H_{A}), 2.33 (s, 3H, CH₃); 2.33 (m, 2H, NC_{4′}H_{B}, NC₆H_{B}); 2.09-2.06 (m, 2H, C₄H_{B}, C₃H_{A}); 1.97 (m, 1H, C₃H_{B}); 1.89 (m, 2H, C_{3′}H, C₇H); 1.70 (s, 2H, C_{9′}H₂); MS (ESI): m/z=432.2 (M+1)⁺

### Example 13

### (5S)-5-(7-benzyl-3,7-diazabicyclo[3.3.1]nonane-3-carbonyl)-1-(2,6-dichlorobenzyl)pyrrolidin-2-one (1g)

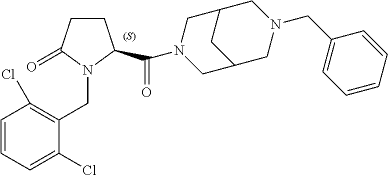

The compound was prepared from N-(2,6-dichlorobenzyl) pyroglutamic acid as described in the case of 1f

Yield=76%; MP: 50-55° C.; [α]_{D}=+51.6304 (Chloroform, c=0.10);

¹H NMR (300 MHz, CDCl₃, ppm); δ7.35-7.24 (m, 8H, 2×Ph), 5.31-5.26 (d, 1H, PhCH_{A}), 4.60-4.56 (d, 1H, C(O)NC_{2′}H_{A}), 4.46-4.41 (d, 1H, PhCH_{B}), 4.05-4.02 (dd, 1H, NC₂H), 3.50-3.46 (m, 2H, PhCH_{A′}, NC_{8′}H_{A}), 3.28-3.24 (m, 2H, PhCH_{B}, C(O)NC_{4′}H_{A}), 3.05-3.01 (m, 1H, C(O)NC_{2′}H_{B}), 2.90-2.87 (m, 2H, C(O)NC_{8′}H_{B}, NC_{6′}H_{A}), 2.56 (m, 1H, C₄H_{A}), 2.36 (m, 2H, C_{4′}H_{B}, NC_{6′}H_{B}), 2.11-2.01 (m, 2H, C₄H_{B}, C₃H_{A}), 1.99-1.97 (m, 1H, C₃H_{B}), 1.93 (m, 2H, C_{3′}H, C_{7′}H), 1.73 (bs, 2H, C_{9′}H₂); ¹³C NMR (50 MHz, CDCl₃, ppm) δ175.10 (CON(CH₃)₂), 168.25 (C═O), 151.52, 137.89, 129.57 (Ph), 128.58 (Ph), 128.45 (Ph), 128.33 (Ph), 127.26 (Ph), 127.01 (Ph), 63.54 (NCH₂Ph), 59.53 (C_{6′}), 58.47 (C_{4′}), 56.20 (NCH₂), 46.45 (NC_{2′}), 31.08 (bridge CH₂), 29.62 (C₄), 29.30 (C_{3′}), 28.55 (C_{7′}), 21.81 (CH₂); IR (KBr): 3639.2, 3400, 2955, 2800, 1690, 1645, 1439, 1362, 1228, 1154, 1119 cm⁻¹; MS (ESI): m/z=486.3 (M+).

### Example 14

### (5S)-5-(7-benzyl)-3,7-diazabicyclo[3.3.1]nonane-3-carbonyl)-1-(4-chlorobenzyl)pyrrolidin-2-one (1h)

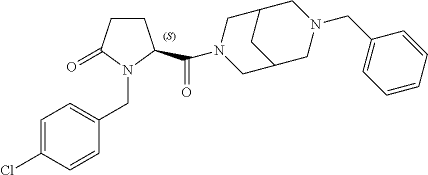

The compound was prepared from N-(4-chlorobenzyl)pyroglutamic acid as described in the case of 1f

Yield=67%; ¹H NMR (300 MHz, CDCl₃, ppm) δ7.45-7.04 (m, 8H, Ph-H) 5.20-5.05 (d, 1H, PhCH_{A}), 4.61-4.56 (d, 1H, C(O)NC_{2′}H_{A}), 4.20-4.12 (dd, 1H, NC₂H), 3.83-3.71 (d, 1H, PhCH_{B}), 3.52-3.41 (m, 2H, PhCH_{A′}, NC_{8′}H_{A}), 3.19-3.15 (m, 2H, PhCH_{B′}, C(O)NC_{4′}H_{A}), 2.95 (m, 2H, C(O)NC₂, H_{B}, NC_{8′}H_{B}), 2.65 (m, 1H, NC_{6′}H_{A}), 2.15-1.88 (m, 4H, C_{4′}H_{B}, NC_{6′}H_{B} C₄H_{B}, C₃H_{A}), 1.83-1.79 (m, 1H, C₃H_{B}), 1.74 (m, 2H, C_{3′}H, C_{7′}H, bs, 2H, C_{9′}H₂); ¹³C NMR (50 MHz, CDCl₃, ppm); 175.33, 168.44, 137.22, 136.79, 133.38, 131.46, 130.16, 129.28, 128.54, 127.89, 120.79, 62.72, 59.36, 58.16, 49.20, 46.33, 45.04, 31.05, 29.96, 29.68, 29.40, 29.19, 28.48, 21.10; IR (KBr): 3870,3777, 3588, 3526, 2924, 2276, 1680, 1451, 1220 cm⁻¹

### Example 15

### (5S)-5-(7-benzyl-3,7-diazabicyclo[3.3.1]nonane-3-carbonyl)-1-tosylpyrrolidin-2-one, (1i)

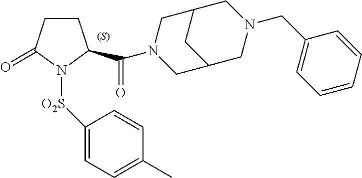

The compound was prepared from p-toluene sulphonic acid as described in the case of 1f

Yield=83%; ¹H NMR (300 MHz, CDCl₃, ppm) δ7.81-7.79 (d, 2H, SO₂Ph) 7.31-7.21 (m, 7H, SO₂Ph, Ph), 4.84-4.81 (d, 1H, C(O)NC_{2′}H_{A}), 3.86-3.82 (d, 1H, PhCH_{A′}), 3.56-3.32 (m, 4H, PhCH_{B′}, C(O)NC_{4′}H_{A}, NC_{8′}H_{A}, NC₂H), 3.01-2.89 (m, 3H, C(O)NC_{2′}H_{B}, C(O)NC_{8′}H_{B}, NC_{6′}H_{A}), 2.43 (s, 3H, CH₃), 0.2.34-2.31 (d, 1H, C₃H_{A}), 2.17-1.69 (m, 10H, C_{4′}H_{B}, NC_{6′}H_{B}, C_{3′}H, C_{7′}H, C₃H_{B}, C₄H, C₅H, C_{9′}H₂); ¹³C NMR (200 MHz, CDCl₃, ppm) δ175.61, 168.19, 163.33, 139.25, 129.83, 128.77, 128.62, 128.33, 127.05, 63.53, 59.34, 58.36, 56.57, 49.22, 46.46, 31.91, 29.79, 29.14, 22.67, 21.58, 14.08; IR (KBr): 3783, 3448, 3374, 2923, 2361, 2135, 1817, 1640, 1446, 1337, 1224, 1098 cm⁻¹; MS (ESI): m/z=468.3 (M⁺)

### Example 16

### tert-butyl-7-((S)-1-(4-cyanobenzyl)-5-oxopyrrolidine-2-carbonyl)3,7diazabicyclo[3.3.1]nonane-3-carboxylate, (1j)

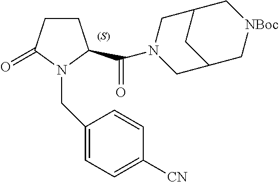

The compound was prepared from N-(4-cyanobenzyl)pyroglutamic acid as described in the case of 1f

Yield=53%; MP: 160-165° C.; ¹H NMR (300 MHz, CDC₃, ppm) δ7.64-7.61 (m, 2H, Ph), 7.28 (m, 2H, Ph) 5.17-5.12 (d, 1H, PhCH_{A}), 4.59-4.55 (d, 1H, C(O)NC_{2′}H_{A}), 4.12-3.92 (d, 1H, PhCH_{B}), 3.87 (d, 1H, NC₂H), 3.12 (m, 1H, PhCH_{A′},), 3.10-3.01 (m, 2H, PhCH_{B′}, C(O)NC_{4′}H_{A}), 2.60-2.51 (m, 1H, C(O)NC_{2′}H_{B}), 2.40-2.50 (m, 2H, C(O)NC_{8′}H_{B}, NC_{6′}H_{A}), 2.12 (m, 1H, C₄H_{A}), 2.10-1.90 (m, 2H, C_{4′}H_{B}, NC_{6′}H_{B}, NC_{8′}H_{A}), 1.90-1.82 (m, 2H, C₄H_{B}, C₃H_{A}), 1.99-1.97 (bs, 4H, C₃H_{B}, C_{3′}H, C_{7′}H, C_{9′}H₂), 1.43 (s, 9H, (CH₃)₃); ¹³C NMR (50 MHz, CDCl₃, ppm) δ175.87 (CON(CH₃)₂), 169.33 (C═O), 154.89 (Ph), 142.20 (Ph), 132.46 (Ph), 128.87 (Ph), 127.58 (Ph), 111.52 (Ph), 79.93 (NCH₂Ph), 56.99 (C_{6′}), 49.56 (C_{4′}), 46.54 (NCH₂), 45.20 (NC_{2′}), 30.27 (bridge CH₂), 29.69 (C₄), 29.52 (C_{3′}), 28.56 (C_{7′}), 28.38 (CH₂) 27.74, 27.32, 23.09; IR (KBr): 3896, 3744, 3700, 3576, 3456, 2924, 2859, 2361, 2228, 1679, 1418 cm⁻¹; MS (ESI): m/z=452.5 (M⁺)

### Example 17

### tert-butyl 7-((S)-1-(4-chlorobenzyl)-5-oxopyrrolidine-2-carbonyl)-3,7-diazabicyclo [3.3.1]nonane-3-carboxylate, (1k)

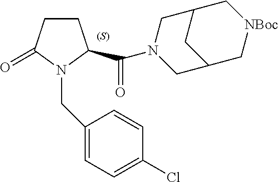

The compound was prepared from N-(4-chlorobenzyl)pyroglutamic acid as described in the case of 1f

Yield=77%; ¹H NMR (300 MHz, CDCl₃, ppm) δ7.30-7.28 (m, 2H, Ph), 7.27-7.12 (m, 2H, Ph) 5.11-5.04 (d, 1H, PhCH_{A}), 4.59-4.54 (d, 1H, C(O)NC_{2′}H_{A}), 4.20-4.00 (d, 1H, PhCH_{B}, NC_{8′}H_{A}), 3.79 (d, 1H, NC₂H), 3.56 (m, 1H, PhCH_{A′},), 3.09-3.04 (m, 2H, PhCH_{B}, C(O)NC_{4′}H_{A}), 2.99-2.96 (m, 1H, C(O)NC_{2′}H_{B}), 2.91-2.86 (m, 2H, C(O)NC_{8′}H_{B}, NC_{6′}H_{A}), 2.50-2.48 (m, 1H, C₄H_{A}), 2.44-2.43 (m, 2H, C_{4′}H_{B}, NC_{6′}H_{B},), 2.41-2.40 (m, 2H, C₄H_{B}, C₃H_{A}), 1.94 (bs, 2H, C₃H_{B}, C₃′H,) 1.81 (bs, 2H, C₇H, C_{9′}H₂), 1.41 (s, 9H, (CH₃)₃);

¹³C NMR (50 MHz, CDCl₃, ppm); 175.57, 169.53, 154.93, 134.94, 133.46, 129.87, 128.78, 79.58, 56.5, 56.49, 56.47, 49.54, 44.70, 29.81, 28.35, 27.74, 22.92; IR(KBr): 3869, 3759, 3496, 3010, 2926, 2860, 1679, 1423 cm⁻¹; MS(ESI): m/z=461.9 (M⁺)

### Example 18

### tert-butyl 7-((S)-1-(2,6-dichlorobenzyl)-5-oxopyrrolidine-2-carbonyl)-3,7-diazabicyclo [3.3.1]nonane-3-carboxylate, (1l)

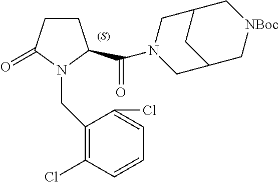

The compound was prepared from N-(2,6-dichlorobenzyl)pyroglutamic acid as described in the case of 1f

Yield=63%; MP: 160-165° C.; ¹H NMR (300 MHz, CDCl₃, ppm) δ7.64-7.61 (m, 2H, Ph), 7.28 (m, 2H, Ph) 5.17-5.12 (d, 1H, PhCH_{A}), 4.59-4.55 (d, 1H, C(O)NC_{2′}H_{A}), 4.12-3.92 (d, 1H, PhCH_{B}), 3.87 (d, 1H, NC₂H), 3.12 (m, 1H, PhCH_{A′},), 3.10-3.01 (m, 2H, PhCH_{B′}, C(O)NC_{4′}H_{A}), 2.60-2.51 (m, 1H, C(O)NC_{2′}H_{B}), 2.40-2.50 (m, 2-1, C(O)NC_{8′}H_{B}, NC_{6′}H_{A}), 2.12 (m, 1H, C₄H_{A}), 2.10-1.90 (m, 2H, C_{4′}H_{B}, NC₆H_{B}, NC_{8′}H_{A}), 1.90-1.82 (m, 2H, C₄H_{B}, C₃H_{A}), 1.99-1.97 (bs, 4H, C₃H_{B}, C_{3′}H, C_{7′}H, C_{9′}H₂), 1.43 (s, 9H, (CH₃)₃); ¹³C NMR (50 MHz, CDCl₃, ppm) δ175.87 (CON(CH₃)₂), 169.33 (C═O), 154.89 (Ph), 142.20 (Ph), 132.46 (Ph), 128.87 (Ph), 127.58 (Ph), 111.52 (Ph), 79.93 (NCH₂Ph), 56.99 (C_{6′}), 49.56 (C_{4′}), 46.54 (NCH₂), 45.20 (NC_{2′}), 30.27 (bridge CH₂), 29.69 (C₄), 29.52 (C_{3′}), 28.56 (C_{7′}), 28.38 (CH₂) 27.74, 27.32, 23.09; IR (KBr): 3896, 3744, 3700, 3576, 3456, 2924, 2859, 2361, 2228, 1679, 1418 cm⁻¹;

### Example 19

### tert-butyl-7-((S)-1-(4-methoxybenzyl)-5-oxopyrrolidine-2-carbonyl)-3,7-diazabicyclo[3.3.1]nonane-3-carboxylate, (1m)

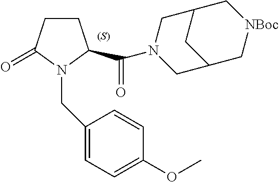

The compound was prepared from N-(4-methoxybenzyl)pyroglutamic acid described in the case of 1f

Yield=54%; ¹H NMR (300 MHz, CDCl₃, ppm) δ7.28-7.23 (m, 2H, Ph), 6.83-6.72 (m, 2H, Ph), 5.14-5.10 (d, 1H, PhCH_{A}), 4.61-4.56 (d, 1H, C(O)NC_{2′}H_{A}), 4.20-4.04 (d, 2H, PhCH_{B}, NC₂H), 3.85-3.75 (s, 3H, OCH₃), 3.74-3.71 (m, 1H, PhCH_{A′},), 3.60-3.48 (m, 1H, PhCH_{B′},), 3.04-3.00 (m, 2H, C(O)NC_{2′}H_{B}, C(O)NC_{4′}H_{A}), 2.94-2.88 (m, 1H, C(O)NC_{8′}H_{B}, NC_{8′}H_{A}), 2.49-2.55 (m, 2H, C₄H_{A}, NC_{6′}H_{A}), 2.31-2.25 (m, 2H, C_{4′}H_{B}, NC_{6′}Ha), 2.20 (m, 2H, C₄H_{B}, C₃H_{A}), 1.80 (bs, 4H, C₃H_{B}, C_{3′}H, C_{7′}H, C_{9′}H₂), 1.42 (s, 9H, (CH₃)₃);

¹³C NMR (50 MHz, CDCl₃, ppm) 172.17, 169.73, 159.90, 139.16, 137.77, 129.58, 120.78, 114.11, 114.03, 113.96, 113.22, 113.05, 79.74, 55.21, 55.18, 49.49, 45.31, 33.76, 29.63, 28.32, 22.81, 14.07; IR (KBr): 3900, 3565, 3366, 3013, 2926, 2856, 2196, 1679, 1434, 1363, 1219 cm⁻¹; MS (ESI): m/z=457.5 (M⁺)

### Example 20

### tert-butyl-7-((S)-1-(naphthalen-1-ylmethyl)-5-oxopyrrolidine-2-carbonyl)-3,7-diazabicyclo [3.3.1]nonane-3-carboxylate, (1n)

The compound was prepared from N-(1-naphthyl)pyroglutamic acid as described in the case of 1f

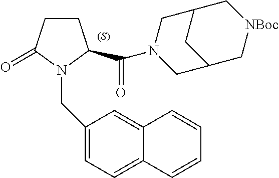

Yield=77%; ¹H NMR (300 MHz, CDCl₃, ppm) δ8.05-7.37 (m, 7H, Naphthyl), 5.62-5.67 (d, 1H, PhCH_{A}), 5.04-4.97 (d, 1H, PhCH_{B}), 4.62-4.52 (d, 1H, C(O)NC_{2′}H_{A}), 4.11-4.07 (m, 1H, NC₂H, NC_{8′}H_{A}), 3.77-3.76 (m, 1H, PhCH_{A′}), 3.24-3.20 (m, 1H, PhCH_{B′}), 3.01-2.91 (m, 3H, C(O)NC_{4′}H_{A}, C(O)NC_{8′}H_{B}, C(O)NC_{2′}H_{B}), 2.66-2.42 (m, 3H, NC_{6′}H_{A} 1H, C₄H_{A}, C_{4′}H_{B}), 2.39 (m, 1H, NC_{6′}H_{B}), 2.07-2.03 (m, 5H, C₄H_{B}, C₃H_{A}, C₃H_{B}, C_{3′}H, C_{7′}H), 1.70 (bs, 2H, C_{9′}H₂), 1.42 (s, 9H, (CH₃)₃); IR (KBr): 3947, 3675, 3484, 3421, 3287, 2923, 2853, 2361, 1674, 1452, 1365 cm⁻¹; MS (ESI): m/z=447.5 (M⁺)

### Example 21

### (5S)-5-(7-(4-benzyl)-3,7-diazabicyclo[3.3.1]nonane-3-carbonyl)-1-(4-bromobenzyl)pyrrolidin-2-one, (1o)

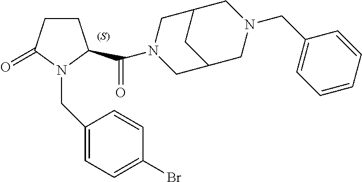

The compound was prepared from N-(4-bromobenzyl)pyroglutamic acid as described in the case of 1f

Yield=65%; ¹H NMR (300 MHz, CDCl₃, ppm) δ7.45-7.04 (m, 8H, Ph-H) 5.20-5.15 (d, 1H, PhCH_{A}), 4.61-4.56 (d, 1H, C(O)NC_{2′}H_{A}), 4.20-4.12 (dd, 1H, NC₂H), 3.83-3.71 (d, 1H, PhCH_{B}), 3.52-3.41 (m, 2H, PhCH_{A′}, NC_{8′}H_{A}), 3.19-3.15 (m, 2H, PhCH_{B′}, C(O)NC_{4′}H_{A}), 2.95 (m, 2H, C(O)NC_{2′}H_{B}, NC_{8′}H_{B}), 2.65 (m, 1H, NC_{6′}H_{A}), 2.15-1.88 (m, 4H, C_{4′}H_{B}, NC_{6′}H_{B}, C₄H_{B}, C₃H_{A}), 1.83-1.79 (m, 1H, C₃H_{B}), 1.74 (m, 2H, C_{3′}H, C_{7′}H, bs, 2H, C_{9′}H₂);

¹³C NMR (50 MHz, CDCl₃, ppm); 175.33, 168.44, 137.22, 136.79, 133.38, 131.46, 130.16, 129.28, 128.54, 127.89, 120.79, 62.72, 59.36, 58.16, 49.20, 46.33, 45.04, 31.05, 29.96, 29.68, 29.40, 29.19, 28.48, 21.10; IR (KBr): 3870,3777, 3588, 3526, 2924, 2276, 1680, 1451, 1220 cm⁻¹; MS (ESI): m/z=510.3 (M⁺)

### Example 22

### (5S)-5-(7-benzyl)-3,7-diazabicyclo[3.3.1]nonane-3-carbonyl)-1-(4-methoxybenzyl) pyrrolidin-2-one, (1p)

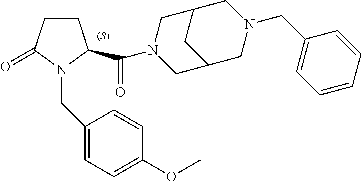

The compound was prepared from N-(4-methoxybenzyl)pyroglutamic acid as described in the case of 1f

Yield=75%; ¹H NMR (300 MHz, CDCl₃, ppm) δ7.55-7.08 (m, 8H, Ph-H) 5.19-5.16 (d, 1H, PhCH_{A}), 4.59-4.55 (d, 1H, C(O)NC_{2′}H_{A}), 4.22-4.09 (dd, 1H, NC₂H), 3.83-3.70 (d, 1H, PhCH_{B}), 3.74 (s, 3H, OCH₃), 3.50-3.39 (m, 2H, PhCH_{A′}, NC_{8′}H_{A}), 3.20-3.16 (m, 2H, PhCH_{B′}, C(O)NC_{4′}H_{A}), 2.95 (m, 2H, C(O)NC_{2′}H_{B}, NC_{8′}H_{B}), 2.64 (m, 1H, NC_{6′}H_{A}), 2.17-1.87 (m, 4H, C_{4′}H_{B}, NC_{6′}H_{B} C₄H_{B}, C₃H_{A}), 1.83-1.79 (m, 1H, C₃H_{B}), 1.76-1.57 (m, 2H, C_{3′}H, C₇H, bs, 2H, C_{9′}H₂); ¹³CNMR (50 MHz, CDCl₃, ppm); 175.30, 167.45, 137.22, 138.79, 134.38, 131.40, 131.16, 130.28, 128.55, 127.80, 121.79, 62.62, 59.26, 58.21, 49.20, 46.42, 45.00, 31.17, 30.22, 29.68, 29.42, 29.20, 28.50, 21.12; IR (KBr): 3872, 3775, 3584, 3540, 2934, 2277, 1668, 1453, 1222 cm⁻¹;

### Example 23

### 5-(7-Benzoyl-3,7-diaza-bicyclo[3.3.1]nonane-3-carbonyl)-1-(2-bromo-benzyl)-pyrrolidin-2-one, (1q)

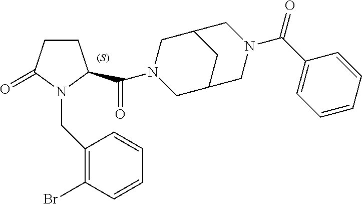

Step-1: 1b (1.5 g, 1.0 eq., 4.39 mmoles) was weighed and dissolved in dry DCM (10 ml). To the stirred solution at a temperature of 0° C., TFA (1.641 ml, 5.0 eq., 2.196 mmoles) was injected slowly and allowed to stir for 1 hour. The resulting mixture was extracted with DCM. It was washed with water and brine. Organic layer was collected and the combined fractions were dried over anhydrous sodium sulphate and concentrated to get yellow oily liquid (1.2 g).

Step-2: Benzoyl chloride (0.104 ml, 1.2 eq, 0.741 mmol) was added drop wise to the stirring solution of crude mass in DCM from step-1 (250 mg, 1 eq, 0.617 mmol) and triethylamine (0.198 ml, 2.3 eq, 1.42 mmol) in dry dichloromethane at 0° C. and allowed to stir for half hour. The reaction mixture was washed with 1N HCl (1×25 ml), 20% NaHCO₃ (1×25 ml). The combined organics were washed with anhydrous sodium sulphate and concentrated to obtain yellow oily liquid. The crude product was purified by column chromatography on silica (Chloroform: Methanol, 8:2) to obtain the pure product

Yield: 88%; MP: 85° C.; [α]_{D}^{27° C.}: +13.73 (Methanol, c=0.1000); IR (KBr): 3404.0, 2929.5, 2365.0, 1629.8, 1429.4, 1351.7, 1246.7, 1085.7 cm⁻¹; ¹H NMR (300 MHz, CDCl₃, ppm) δ7.57-7.17 (m, 9H, 2×Ph); 5.15-5.10 (br d, J=15 Hz, 1H, PhCH_{A}); 4.80-4.75 (br d, J=15 Hz, 1H, NC_{2′}H_{A}); 4.62-4.57 (br d, J=15 Hz, 1H, PhCH_{B}); 4.22-4.11 (m, 1H, NC₂H); 3.88-3.83 (d, J=15 Hz, 1H, NC_{8′}H_{A}); 3.71-3.66 (d, J=15 Hz, 1H, NC_{2′}H_{B}); 3.24-3.12 (m, 3H, NC_{8′}H_{B}, NC_{4′}H_{A}, NC_{6′}H_{A}); 2.90-2.86 (d, J=12 Hz, NC_{6′}H_{B}); 2.52-2.49 (m, 1H, C₄H_{A}); 2.44-2.40 (m, 1H, C_{4′}H_{B}); 2.34-2.24 (m, 1H, C₄H_{B}); 2.22-2.07 (m, 1H, C₃H_{A}); 1.95-1.91 (m, 3H, C₃H_{B}, C_{3′}H, C_{7′}H); 1.83 (m, 2H, C_{9′}H₂); ¹³C NMR (75 MHz, CDCl₃, ppm) δ175.83 (C═O), 171.35 (N_{1′}CO), 170.21 (N_{5′}CO), 135.87 (Ph), 132.80 (Ph), 131.37 (Ph), 129.43 (Ph), 128.74 (Ph), 127.84 (Ph), 126.70 (Ph), 124.14 (Ph), 56.95 (NC₂), 52.47 (NC_{2′}), 49.53 (NC_{8′}), 46.56 (NC_{6′}), 46.08 (NC_{4′}), 45.30 (NCH₂), 30.85 (Bridge CH₂) 29.87 (C₃), 27.66 (C_{3′}, C_{7′}), 23.39 (C₄); MS (ESI): m/z=512 (M+3)⁺

### Example 24

### 1-(2-Bromo-benzyl)-5-[7-(toluene-4-sulphonyl)-3,7-diaza-bicyclo[3.3.1]nonane-3-carbonyl]-pyrrolidin-2-one, (1r)

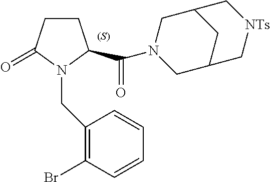

p-Toluene sulphonyl chloride (0.140g, 1.2 eq, 0.739 mmol) was added drop wise to the stirring solution of Boc de-protected product of 1b (250 mg, 1 eq, 0.616) and TEA (0.197 ml, 2.3 eq, 1.41 mmol) in dry DCM at 0° C. and allowed to stir for half hour. The reaction mixture was washed with 1N HCl (1×25 ml), 20% NaHCO₃ (1×25 ml). The combined organics were washed with anhydrous sodium sulphate and concentrated to obtain yellow oily liquid.

Yield=85%; [α]_{D}^{27° C.}: −2.8263 (Methanol, c=0.1000); MP: 203-205° C.; IR (KBr): 3451.8, 1638.4 cm⁻¹; ¹H NMR (300 MHz, CDCl₃, ppm): δ 7.58-7.15 (m, 8H, 2×Ph); 5.13-5.08 (d, J=15 Hz, 1H, PhCH_{A}); 4.66-4.62 (d, J=12 Hz, 1H, NC_{2′}H_{A}); 4.27-4.23 (m, 1H, NC₂H); 4.15-4.10 (d, J=15 Hz, 1H, PhCH_{B}); 3.793.76 (d, J=9 Hz, 2H, NC_{8′}H_{A}, NC_{2′}H_{B}); 3.66-3.62 (d, J=12 Hz, 1H, NC_{4′}H_{A}); 3.20-3.15 (m, 1H, NC_{8′}H_{B}); 2.96-2.91 (m, 1H, NC_{6′}H_{A}); 2.71-2.62 (m, 1H, NC₄H_{A}); 2.47-2.43 (m, 6H, NC_{4′}H_{B}, NC_{6′}H_{B}, C₄H_{B}, CH₃); 2.34-2.31 (m, 2H, C₃H₂); 2.28-2.05 (C_{3′}H, C_{7′}H); 1.98 ppm (m, 2H, C_{9′}H₂); ¹³C NMR (75 MHz, CDCl₃, ppm): δ 176.05 (C═O), 169.32 (C═O), 143.74 (Ph), 135.97 (Ph), 132.80 (Ph), 131.50 (Ph), 131.12 (Ph), 129.67 (Ph), 129.33 (Ph), 127.79 (Ph), 127.75 (Ph), 124.06 (Ph), 56.80 (NC₂), 50.59 (NC_{8′}), 48.65 (NC_{2′}), 46.13 (NC_{6′}), 45.38 (NCH₂), 29.74 (Bridge CH₂), 27.73 (NC_{4′}), 27.28 (C_{3′}, C_{4′}) 22.57 (CH₃), 21.51 ppm (C₄); MS (ESI): m/z: 562.0 (M+1)⁺

### Example 25

### 5-(7-Benzoyl-3,7-diaza-bicyclo[3.3.1]nonane-3-carbonyl)-1-(4-methyl-benzyl)-pyrrolidin-2-one, (1s)

Benzoyl chloride (0.123 g, 1.2 eq, 0.880 mmol) was added drop wise to the stirring solution of Boc deprotected product of 1e (250 mg, 1 eq, 0.733 mmol) and TEA (0.235 ml, 2.3 eq, 1.68 mmol) in dry DCM at 0° C. and allowed to stir for half hour. The reaction mixture was washed with 1N HCl (1×25 ml) 20% NaHCO₃ (1×25 ml). The combined organics were washed with anhydrous sodium sulphate and concentrated to obtain yellow oily liquid.

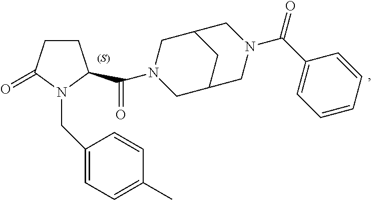

Yield=89.2%; [α]_{D}^{27° C.}: +2.1583 (Methanol, c=0.1000); IR (Neat): 3420.3, 2958.5, 1678.4, 1632.11, 1438.3, 1220.2 cm⁻¹; ¹H NMR (300 MHz, CDCl₃, ppm): δ7.42-7.00 (m, 9H, 2×Ph); 5.15-5.04 (m, 1H, PhCH_{A}); 4.81-4.55 (m, 1H, NC_{2′}H_{A}); 4.16 (m, 1H, NC₂H); 3.89-3.77 (m, 1H, NC_{8′}H_{A}); 3.67-33.64 (m, 1H, PhCH_{B}); 3.33-3.19 (m, 2H, NC_{2′}H_{B}, NC_{8′}H_{B}); 3.04-3.02 (m, 1H, NC_{4′}H_{A}); 2.93-2.89 (m, 7H, C₄H_{A}, NC_{6′}H_{B}, C₄H_{B}, C_{4′}H_{B}, CH₃); 2.22-2.18 (m, 2H, C₃H₂); 1.95-1.81 (m, 2H, C_{3′}H, C_{7′}H); 1.27-1.26 (m, 2H, C₉H₂); ¹³C NMR (75 MHz, CDCl₃, ppm): δ 175.83 (C═O), 171.28 (C═O), 170.20 (NC_{5′}O), 137.4 (ipso Ph), 136.08 (Ph), 136.00 (Ph), 129.40 (Ph), 129.22 (Ph), 128.87 (Ph), 128.72 (Ph), 128.65 (Ph), 128.56 (Ph), 128.20 (Ph), 127.22 (Ph), 126.76 (Ph), 57.49 (NC₂), 49.47 (NCH₂), 46.49 (NC_{4′}), 46.16 (NC_{6′}), 45.92 (NC_{2′}), 45.13 (NC_{8′}), 34.27 (Bridge CH₂), 30.40 (C₄), 27.61 (C_{3′}), 23.40 (C_{7′}), 21.29 (C₃), 21.17 (CH₃); MS (ESI): m/z=446.1 (M+H)⁺

### Example 26

### 1-(4-Methyl-benzyl)-5-[7-(toluene-4-sulphonyl)-3,7-diaza-bicyclo[3.3.1]nonane-3-carbonyl]-pyrrolidin-2-one, (1t)

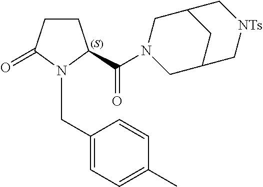

The compound was prepared by the addition of p-toluene sulphonyl chloride (0.167g, 1.2 eq, 0.880 mmol) to the stirring solution Boc de-protected product of 1e (250 mg, 1 eq, 0.733 mmol) and TEA (0.235 ml, 2.3 eq, 1.68 mmol) in dry DCM.

Yield=91.5%; [α]_{D}^{27° C.}: −0.9313 (Methanol, c=0.1000); IR (Neat): 3449.8, 2953.7, 1641.5, 1443.2, 1220.9 cm⁻¹; ¹H NMR (300 MHz, CDCl₃, ppm): δ 7.60-7.00 (m, 8H, 2×Ph), 5.20-5.03 (m, 1H, PhCH_{A}), 4.68-4.65 (m, 1H, NC_{2′}H_{A}), 4.24-4.23 (m, 1H, NC₂H), 3.94-3.64 (m, 3H, PhCH_{B}, NC_{2′}H_{B}, NC_{8′}H_{A}), 3.13-2.97 (m, 2H, NC_{8′}H_{B}, NC_{4′}H_{A}), 2.77-2.76 (m, 1H, NC_{6′}H_{B}), 2.72-2.66 (m, 3H, C₄H_{A}, NC_{6′}H_{B}, C_{4′}H_{B}), 2.45-2.07 (m, 5H, C₄H_{A}, C₃H₂, CH₃), 1.92 (m, 2H, C_{3′}H), 1.66 (m, 1H, C_{7′}H), 1.28-1.25 (m, 2H, C_{9′}H₂); ¹³C NMR (50 MHz, CDCl₃, ppm): δ 151.53 (Ph), 135.80 (Ph), 129.68 (Ph), 129.22 (Ph), 128.51 (Ph), 128.25 (Ph), 128.65 (Ph), 128.56 (Ph), 128.20 (Ph), 127.22 (Ph), 126.76 (Ph), 46.10 (NCH₂), 45.01 (NC₂), 34.20 (Bridge CH₂), 34.23 (NC_{4′}), 30.34 (NC_{6′}), 27.80 (C_{3′}), 27.36 (C_{7′}), 22.64 (CH₃), 21.53 (CH₃), 21.20 (C₃); MS (ESI): m/z: 496.0 (M+H)⁺

### Example 27

### (5S)-5-(2-bromobenzyl-3,7-diazabicyclo[3.3.1]nonane-3-carbonyl)-1-(4-methylbenzyl)pyrrolidin-2-one, (1u)

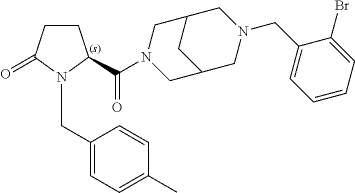 [SMILES: Cc1ccc(CN2C(=O)CC[C@H]2C(=O)N2CC3CC(CN(Cc4ccccc4Br)C3)C2)cc1]

(5S)-5-(3,7-diazabicyclo[3.3.1]nonane-3-carbonyl)-1-(4-methylbenzyl)pyrrolidin-2-one (250 mg, 1.0 eq., 0.76 mmol) was weighed and taken in round bottom flask, dissolved in dry acetone (2 ml). 2 g of anhydrous potassium carbonate (K₂CO₃) was added. 2-bromobenzyl bromide (182.4 mg, 1.5 eq., 1.14 mmol) was added to the reaction mixture and refluxed in an oil bath at 50-60° C. for 2 hours with stirring. The reaction was monitored for completion by TLC. After the completion of reaction, reaction mixture was filtered to remove K₂CO₃ and concentrated in vacuum. The desired product was isolated from the crude reaction mixture by column chromatography.

Yield=64%; MP: 85-90° C.; ¹H NMR (300 MHz, CDCl₃, ppm) δ7.29 (m, 9H, 2×Ph), 5.18-5.14 (d, 1H, PhCH_{A}), 4.94-4.90 (m, 1H, C(O)NC_{2′}H_{A}), 4.61-4.56 (d, 1H, PhCH_{B}), 4.14 (m, 1H, NC₂H), 3.85-3.80 (m, 2H, PhCH_{A′}, NC_{8′}H_{A}), 3.51-3.47 (m, 2H, PhCH_{B′}, C(O)NC_{4′}H_{A}), 3.29-3.15 (m, 2H, C(O)NC_{2′}H_{B}, C(O)NC_{8′}H_{B},), 3.04-2.89 (m, 1H, NC_{6′}H_{A}), 2.57 (m, 1H, C₄H_{A}), 2.37 (m, 2H, C_{4′}H_{B}, NC_{6′}H_{B}), 2.09-2.08 (m, 2H, C₄H_{B}, C₃H_{A}), 1.98 (m, 1H, C₃H_{B}), 1.93 (m, 2H, C_{3′}H, C_{7′}H), 1.72 (bs, 2H, C_{9′}H₂); ¹³C NMR (50 MHz, CDCl₃, ppm) δ181.36, 180.12, 169.59, 143.12, 129.42, 128.74, 128.17, 127.64, 114.05, 63.58, 59.23, 58.80, 49.77, 48.27, 46.64, 30.88, 29.83, 29.68, 29.33, 21.52; IR (KBr): 3444, 2925, 2857, 2372, 2338, 2141, 1638, 1447, 1355, 1225, 1093 cm⁻¹; MS (ESI): m/z=452.3 (M⁺)

### Example 28

### (5S)-5-(7-(4-bromobenzyl)-3,7-diaza bicyclo[3.3.1]nonane-3-carbonyl)-1-(4-methylbenzyl)pyrrolidin-2-one, (1v)

Please refer the example 1u (4-bromobenzyl bromide used here)

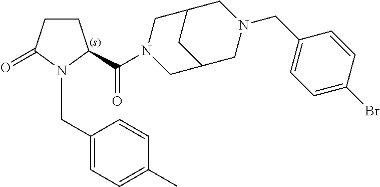 [SMILES: Cc1ccc(CN2C(=O)CC[C@H]2C(=O)N2CC3CC(CN(Cc4ccc(Br)cc4)C3)C2)cc1]

Yield=67%; ¹H NMR (300 MHz, CDCl₃, ppm) δ7.45-7.04 (m, 8H, Ph-H) 5.20-5.15 (d, 1H, PhCH_{A}), 4.61-4.56 (d, 1H, C(O)NC_{2′}H_{A}), 4.20-4.12 (dd, 1H, NC₂H), 3.83-3.71 (d, 1H, PhCH_{B}), 3.52-3.41 (m, 2H, PhCH_{A′}, NC_{8′}H_{A}), 3.19-3.15 (m, 2H, PhCH_{B}, C(O)NC_{4′}H_{A}), 2.95 (m, 2H, C(O)NC_{2′}H_{B}, NC_{8′}H_{B}), 2.65 (m, 1H, NC_{6′}H_{A}), 2.15-1.88 (m, 4H, C_{4′}H_{B}, NC_{6′}H_{B}, C₄H_{B}, C₃H_{A}), 1.83-1.79 (m, 1H, C₃H_{B}), 1.74 (m, 2H, C_{3′}H, C_{7′}H, bs, 2H, C_{9′}H₂)

¹³C NMR (50 MHz, CDCl₃, ppm) 180.33, 173.40, 142.20, 138.34, 137.69, 134.78, 134.30, 133.53, 132.90, 67.65, 64.34, 63.14, 61.36, 59.96, 54.18, 51.32, 50.03, 36.06, 34.68, 34.17, 26.47; IR (KBr): 3891, 3806, 3708, 3625, 3585, 3446, 2924, 2411, 1676, 1452, 1363, 1221, 1091 cm⁻¹; MS (ESI): m/z=510.4 (M⁺)

### Example 29

### (5S)-5-(7-(4-chlorobenzyl)-3,7-diazabicyclo[3.3.1]nonane-3-carbonyl)-1-(4-methylbenzyl)pyrrolidin-2-one, (1w)

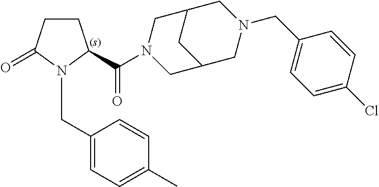 [SMILES: Cc1ccc(CN2C(=O)CC[C@H]2C(=O)N2CC3CC(CN(Cc4ccc(Cl)cc4)C3)C2)cc1]

Please refer the example 1u (4-chlorobenzyl chloride used here)

Yield=62%; ¹H NMR (300 MHz, CDCl₃, ppm) δ: 7.52-7.49 (m, 1H) 7.35-7.28 (m, 2H), 7.11-7.00 (m, 4H) 6.85-6.82 (d, 1H) 5.18-5.13 (d, 1H, PhCH_{A}), 4.64-4.59 (d, 1H, C(O)NC_{2′}H_{A}), 4.16-4.12 (dd, 1H, NC₂H), 3.79-3.74 (d, 1H, PhCH_{B}), 3.60-3.41 (m, 2H, PhCH_{A′}, NC_{8′}H_{A}), 3.16-3.00 (m, 2H, PhCH_{B′}, C(O)NC_{4′}H_{A}), 2.91-2.88 (m, 1H, C(O)NC_{2′}H_{B}), 1.98-1.97 (m, 2H, C(O)NC_{8′}H_{B}, NC_{6′}H_{A}), 1.95-1.93 (m, 2H, C_{4′}H_{B}, NC_{6′}H_{B}), 1.85 (m, 2H, C₄H_{B}, C₃H_{A}), 1.44-1.43 (m, 1H, C₃H_{B}), 1.28 (m, 2H, C_{3′}H, C_{7′}H, bs, 2H, C_{9′}H₂); ¹³C NMR (50 MHz, CDCl₃, ppm) 175.50, 168.55, 137.20, 136.98, 133.44, 132.65, 129.27, 128.54, 62.45, 59.62, 58.58, 56.41, 54.93, 49.15, 46.36, 45.04, 29.90, 29.90, 29.26, 28.51, 21.10; IR (KBr): 3869, 3441, 3013, 2365, 1679, 1515, 1450, 1218 cm⁻¹; MS (ESI): m/z=466.01 (M⁺)

### Example 30

### Benzyl(2S)-1-(7-benzyl-3,7-diazabicyclo[3.3.1]nonan-3-yl)-3-methyl-1-oxobutan-2-yl carbamate, (1x)

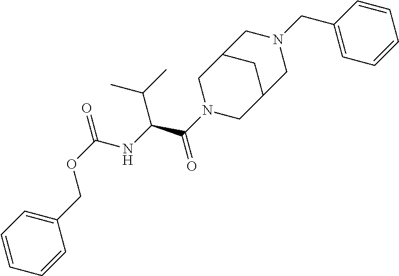 [SMILES: CC(C)[C@H](NC(=O)OCc1ccccc1)C(=O)N1CC2CC(CN(Cc3ccccc3)C2)C1]

Cbz protected L-Valine (530 mg, 1.00 eq., 2.10 mmol) was weighed and taken in a round bottom flask, dissolved in dry DCM (10 ml). At 0° C., HOBt (427 mg, 1.5 eq., 3.10 mmol) was added and allowed to stir for 15 mins. Further 519 mg of DCC (1.2 eq., 2.50 mmol) dissolved in dry DCM was injected slowly to the reaction mixture in a moisture free condition. After 15 minutes 499 mg of N-benzyl bispidine (1.1 eq., 2.32 mmol) dissolved in dry DCM was added slowly to the reaction mixture and allowed to stir for 1-2 hours at 25° C. The reaction was monitored for completion by TLC. After the completion of reaction the reaction mixture was filtered to remove the DCU formed during the reaction ant the washed with 1 N HCl and Sodium bicarbonate solution to remove the excess of unreacted base and acid respectively. The organic layer was collected and evaporated to get the crude product which was purified by column chromatography to obtain the pure product (456 mg) as yellow oily liquid.

Yield=67%; ¹H NMR (300 MHz, CDCl₃, ppm) δ7.37-7.26 (m, 10H, Ph-H) 5.91-5.87 (d, 1H, NH), 5.11-5.05 (d, 3H, C(O)OCH₂, NHCHC(O)) 4.72-4.65 (d, 1H, C(O)NC_{2′}H_{A}), 4.25-4.20 (d, 1H, CH(CH₃)₂) 4.12-4.08 (d, 1H, CONC_{8′}H_{B}), 3.51-3.46 (d, 2H, NCH_{A′}Ph, NCH_{B′}Ph), 3.33 (d, 2H, CHCH_{A}Ph, CHCH_{B}Ph), 3.28-3.02 (m, 3H, CH₃), 2.90 (d, 3H, CH_{3′}), 2.35-2.25 (m, 2H, 2×NC₆H), 2.11-2.98 (m, 4H, C(O)NC_{4′}H_{A}, C(O)NC_{4′}H_{B}, NC_{8′}H_{A}, NC_{8′}H_{B}), 1.87-1.63 (m, 4H, C₁′H, C₅′H, bridge CH₂); ¹³C NMR (200 MHz, CDCl₃, ppm) δ166.51, 156.59, 139.26, 136.42, 128.95, 128.47, 128.37, 128.22, 128.13, 127.99, 66.77, 66.75, 66.73, 63.49, 58.53, 55.56, 50.49, 46.98, 29.70, 22.69, 20.12, 14.12; IR (KBr): 3932, 3780, 3744, 3704, 3666, 3606, 3559, 3483, 3436, 3191, 3093, 2936, 2844, 2383, 2344, 2274, 1634, 1451, 1222, 1107, 1022 cm⁻¹; MS (ESI): m/z=450.3 (M+)

### Example 31

### Benzyl(2S)-1-(7-benzyl-3,7-diazabicyclo[3.3.1]nonan-3-yl)-1-oxo-3-phenylpropan-2-yl carbamate (1y)

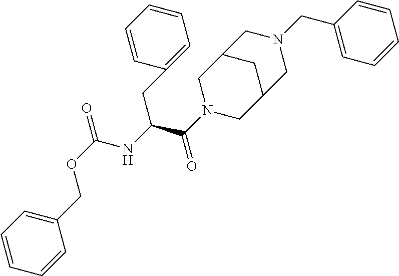 [SMILES: O=C(N[C@@H](Cc1ccccc1)C(=O)N1CC2CC(CN(Cc3ccccc3)C2)C1)OCc1ccccc1]

Cbz protected L-Phenyalanine (580 mg, 1.00 eq., 1.93 mmol) was weighed and taken in a round bottom flask, dissolved in dry DCM (10 ml). At 0° C., HOBt (393 mg, 1.5 eq., 2.90 mmol) was added and allowed to stir for 15 mins. Further 477 mg of DCC (1.2 eq., 2.31 mmol) dissolved in dry DCM was injected slowly to the reaction mixture in a moisture free condition. After 15 min., 459 mg of N-benzyl bispidine (1.1 eq., 2.12 mmol) dissolved in dry DCM was added slowly to the reaction mixture and allowed to stir for 1-2 hours at 25° C. The reaction was monitored for completion by TLC. After the completion of reaction the reaction mixture was filtered to remove the DCU formed during the reaction ant the washed with 1 N HCl and Sodium bicarbonate solution to remove the excess of unreacted base and acid respectively. The organic layer was collected and evaporated to get the crude product which was purified by column chromatography to obtain the pure product (510 mg) as yellow oily liquid.

Yield=53%; ¹H NMR (300 MHz, CDCl₃, ppm) δ7.31-7.16 (m, 15H, Ph-H) 6.06-6.03 (d, 1H, NH), 5.13-5.03 (dd, 3H, C(O)OCH₂, NHCHC(O)) 4.63-4.59 (d, 1H; C(O)NC_{2′}H_{A}), 3.75-3.71 (d, 1H, CONC_{8′}H_{B}), 3.29-3.27 (d, 1H, NCH_{A′}Ph), 3.06-3.04 (d, 1H, NCH_{B′}Ph), 2.81-2.77 (CHCH_{A}Ph), 2.59-2.54 (d, 1H, CHCH_{B}Ph), 2.92-2.77 (m, 4H, C(O)NC_{4′}H_{A}, C(O)NC_{4′}H_{B}, NC_{8′}H_{A}, NC_{8′}H_{B}), 2.19-2.16 (m, 2H, 2×NC₆H), 2.02 (bs, 1H, C₁′H), 1.87 (bs, 1H, C_{5′}H), 1.69 (m, 2H, bridge CH₂); ¹³C NMR (200 MHz, CDCl₃, ppm) 169.33, 155.58, 138.40, 136.99, 129.68, 129.40, 128.94, 128.45, 128.45, 128.22, 126.82, 77.79, 76.52, 66.65, 63.45, 59.29, 58.39, 51.90, 49.84, 46.51, 39.84, 31.61, 29.23, 28.43; IR (KBr): 3865, 3755, 3439, 3295, 1634, 1507, 1453, 1219, 1145, 1049 cm⁻¹; MS (ESI): m/z=498.3 (M⁺)

### Example 32

### Benzyl(2S)-1-(7-benzyl-3,7-diazabicyclo[3.3.1]nonan-3-yl)-4-methyl-1-oxopentan-2-ylcarbamate (1z)

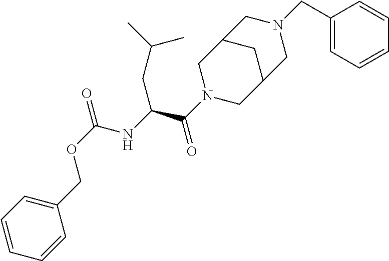 [SMILES: CC(C)C[C@H](NC(=O)OCc1ccccc1)C(=O)N1CC2CC(CN(Cc3ccccc3)C2)C1]

Cbz protected L-Leucine (530 mg, 1.00 eq., 1.93 mmol) was weighed and taken in a round bottom flask, dissolved in dry DCM (10 ml). At 0° C.; HOBt (405 mg, 1.5 eq., 2.90 mmol) was added and allowed to stir for 15 mins. Further 469 mg of DCC (1.2 eq., 2.21 mmol) dissolved in dry DCM was injected slowly to the reaction mixture in a moisture free condition. After 15 mins 452 mg of N-benzyl bispidine (1.1 eq., 2.02 mmol) dissolved in dry DCM was added slowly to the reaction mixture and allowed to stir for 1-2 hours at 25° C. The reaction was monitored for completion by TLC. After the completion of reaction the reaction mixture was filtered to remove the DCU formed during the reaction ant the washed with 1 N HCl and Sodium bicarbonate solution to remove the excess of unreacted base and acid respectively. The organic layer was collected and evaporated to get the crude product which was purified by column chromatography to obtain the pure product (482 mg) as yellow oily liquid.

Yield=63%; ¹H NMR (300 MHz, CDCl₃, ppm) δ7.36 (m, 10H, Ph-H) 5.93-5.90 (d, 1H, NH), 5.72-5.69 (d, 1H, NHCHC(O)), 5.11-5.05 (d, 2H, C(O)OCH₂,), 4.95-4.89 (d, 3H, C(O)NC_{2′}H_{A}, NHCHCH₂), 4.74-4.72 (d, 1H, CH(CH₃)₂), 4.64-4.60 (d, 2H, NCH_{A′}Ph, NCH_{B′}Ph), 4.39-4.34 (d, 1H, CONC_{8′}H_{B}), 4.04-3.99 (d, 1H, CHCH_{A}Ph), 3.84-3.80 (d, 1H, CHCH_{B}Ph), 3.52-3.48 (m, 3H, CH₃), 3.23-3.19 (m, 2H, 2×NC₆H), 2.94 (d, 3H, CH_{3′}), 2.93-2.81 (m, 4H, C(O)NC_{4′}H_{A}, C(O)NC_{4′}H_{B}, NC_{8′}H_{A}, NC_{8′}H_{B}), 2.41-2.38 (d, 1H, C_{1′}H), 2.33-2.29 (d, 1H, C_{5′}H), 2.11 (m, 2H, bridge CH₂); ¹³C NMR (200 MHz, CDCl₃, ppm) 171.17, 156.40, 137.88, 136.61, 128.43, 128.37, 128.25, 127.92, 126.91, 63.52, 59.59, 58.14, 49.98, 46.66, 42.68, 30.73, 29.70, 29.47, 28.93, 24.66, 24.54, 23.79, 23.47, 22.16, 21.84; IR (KBr): 3842, 3756, 3016, 2925, 1711, 1629, 1508, 1453, 1335, 1218, 1118, 1048 cm⁻¹; MS (ESI): m/z=464.3 (M⁺)

### Evaluation of Anti-Thrombotic Activity of Compounds

### In Vivo/Ex Vivo Studies

The animals, male Swiss albino mice (20-25g), were obtained from the National Laboratory Animal Centre of CSIR-Central Drug Research Institute, Lucknow. All the animal experiments were subjected to Institutional Animal Ethical Committee (IAEC) guidelines and were conducted according to the guidelines of Experimental Animal Care issued by the Committee for Purpose of Control and Supervision of Experiments on Animals (CPCSEA). The animals were housed in polypropylene cages and maintained on standard chow diet and water ad libitum and on 12 hr/12 hr light-dark cycle at temperature: 25±2° C., humidity: 45-55% and ventilation: 10-12 exchanges/hr.

### Collagen-Epinephrine Induced Pulmonary Thromboembolism

To assess the antithrombotic efficacy of compounds, mice were grouped into vehicle, aspirin and compound treated groups, and each group included ten animals. Pulmonary thromboembolism was induced by injecting a mixture of collagen (150 μg/ml) and adrenaline (50 μg/ml) into the tail vein to achieve final doses of collagen (1.5 mg/kg) and adrenaline (0.5 mg/kg) to induce hind limb paralysis or death.^{11, 12} Number of test animals killed or paralyzed were evaluated (death/paralysis were employed as endpoint to evaluate antithrombotic agents). The percent protection was calculated by taking the ratio of number of test animals killed or paralyzed to that of total tested animals. Results have been reported as percentage protection, which represents protection against collagen and epinephrine induced thromboembolism and expressed as;

Percent Protection=[1−(Pₜₑₛₜ/P_{control})]×100

Where, Pₜₑₛₜ is the number of animals paralyzed/dead in test compound-treated group, and P_{control} is the total number of animals paralyzed/dead in vehicle treated group. The percent protection refers to the number of animals in compound treated group that were prevented from paralysis/death.

### Results:

After 1 hour of dosing by oral route, 14 compounds showed ˜40-60% protection against collagen plus epinephrine induced pulmonary thromboembolism in mice at 30 μM/kg concentration (in vivo), while the standard antithrombotic drug Aspirin displayed only 40% protection at a dose of 170 μM/kg, which is sufficient enough to cause bleeding complications (Table 1).

### Bleeding Time

Bleeding time in mice was evaluated by the method of Dejana et al. (Thromb Res. 1979; 15:191-7) The tail 2 mm from tip of mice was incised and the blood oozed was soaked on a filter paper, which was monitored at an interval of 10-15 sec till the bleeding stops. The time elapsed from the tip incision to the stoppage of bleeding was determined as the bleeding time. The preferred compound, aspirin (170 μM/kg), Clopidogrel (70 μM/kg) or vehicle was given orally 60 min prior to the tail incision in a group of 5 mice each.

### Results:

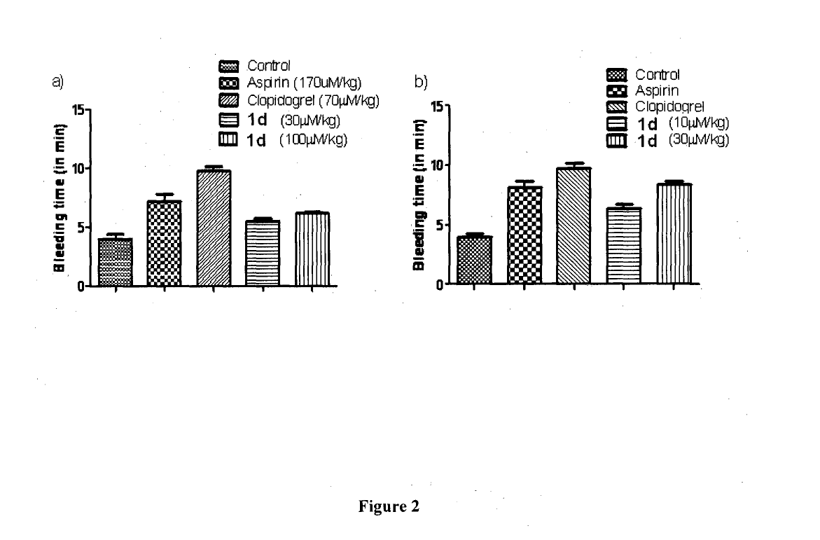

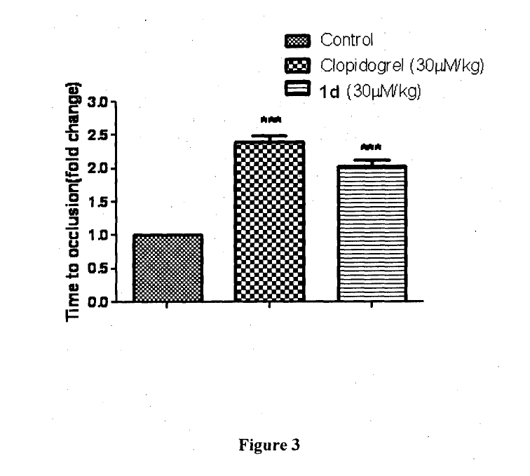

The compound 1d after 1 hr of dosing (by oral route) had a mild effect on bleeding tendency in mice when compared against aspirin and clopidogrel and hence, indicates that the compound escapes the adverse events of bleeding risk in comparison to existing anti-platelet agents, at least in preclinical models. However, after 4 hours (p.o.), the compound 1d (30 μM/kg) displayed upto 60% of protection in collagen-epinephrine induced pulmonary thromboembolism ii mice which was higher than that observed in standard drug Aspirin treated mice (40%). This indicates that the bioavailability and efficacy of compound 1d is increased after 4 hours of oral dosing. The bleeding tendency in 1d treated mice was also increased after 4 hrs (8.4 min) but the prolongation was comparable to that of standard drug Aspirin (8.2 min), and less than Clopidogrel (9.8 min). This suggests that the compound 1d displays a remarkable antithrombotic efficacy much better than the existing anti-platelet drugs, with a moderate alteration in bleeding tendency. (FIG. 2)

### FeCl₃ Induced Thrombosis

Male Swiss albino mice were anesthetized byurethane (1.25 g/kg, i.p.). The carotid artery was carefully dissected and a pulsed Doppler Probe (LDF 100C, BioPac, USA) was placed around it to record the blood flow velocity and patency of the blood vessels. The carotid artery thrombosis was induced by FeCl₃ as follows: a square (1×0.5 mm) of Whatman Chromatography paper was immersed in 10% FeCl₃ solution for 5 min and placed on the carotid artery as described earlier. (Kurz K D, et al., Thromb Res 1990; 60(4):269-80; Surin W R et al J Pharmacol Toxicol Methods. 2010; 61(3):287-91) Thrombosis was monitored as the reduction in carotid artery blood flow. The time at which the blood-flow velocity was decreased to zero was recorded as the time to occlusion (TTO) of the carotid artery. When the blood flow velocity did not occlude within 120 minutes the time to thrombotic occlusion was assigned a value of >120 minutes.

### Results:

FeCl₃ induced thrombosis is one of the widely used animal model for screening of anti-thrombotic agents. The model involves application of FeCl₃ on the adventitial layer of artery to induce vascular injury. FeCl₃ induces the generation of reactive oxygen species that leads to endothelial denudation resulting in platelet adhesion and formation of occlusive platelet rich thrombi. The compound 1d was further evaluated for its antithrombotic efficacy in ferric chloride induced arterial thrombosis model in mice. The compound 1d after 4 hr of its oral administration, prolonged the time to occlusion of carotid artery by 2.2 fold (control 9.5±0.4 min vs 1d 19.2±0.9 min). The standard drug Clopidogrel increased the TTO upto 23±0.9 min. Therefore, the efficacy elicited in this model substantiates the anti-thrombotic potential of this compound (Figure-3).

### In Vitro Studies

From human subjects blood was collected in citrate-phosphate-dextrose (CPD) (1:7) from healthy volunteers (age between 18-60 years) after prior consent. A detailed medical history and physical examination was carried out before phlebotomy. The donors were free from heart, lung, kidney disease, cancer, epilepsy, diabetes, tuberculosis, abnormal bleeding tendency, allergic disease, sexually transmitted diseases, jaundice, malaria, typhoid and thyroid or any other endocrine disorder. Donors were free from any prior medication for last 72 hours.

### Platelet Aggregation Measurements

A turbidimetric method was applied to measure platelet aggregation, using a four channel-Aggregometer (Model 700, Chronolog-corp, Havertown, USA. (Armida P T et al., Thrombosis Research. 1995; 78:107-15, Jain M, Surin W R et al Chem Biol Drug Des. 2012.) Fresh blood was drawn by venipuncture from consenting healthy human volunteers in citrate-phosphate-dextrose. Platelet-rich plasma (PRP) was obtained by centrifugation at 108 g for 20 minutes at 25° C. (Beckman TJ6, USA). Platelet rich plasma (1×10⁸ platelets/ml, 0.45 ml) was pre-warmed to 37° C. for 2 min, then incubated with compound (3-300 μM) or an isovolumetric solvent control (0.5% DMSO) for 5 min before addition of the agonists (i.e., 1 μg/ml Collagen, 5 μM ADP, 25 μM TRAP, 1.5 mg/ml Ristocetin, Arachidonic Acid, collagen related peptide CRP-XL). The reaction was allowed to proceed for at least 5 min, and the extent of aggregation was expressed in percent aggregation by Aggrolink software. (Jain M, Surin W R et al Chem Biol Drug Des. 2012)

### Results:

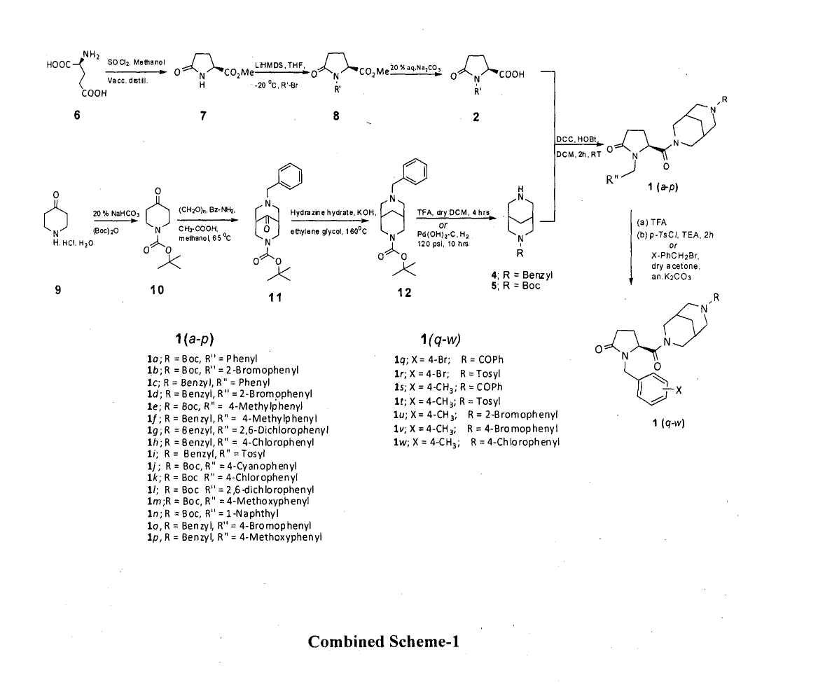

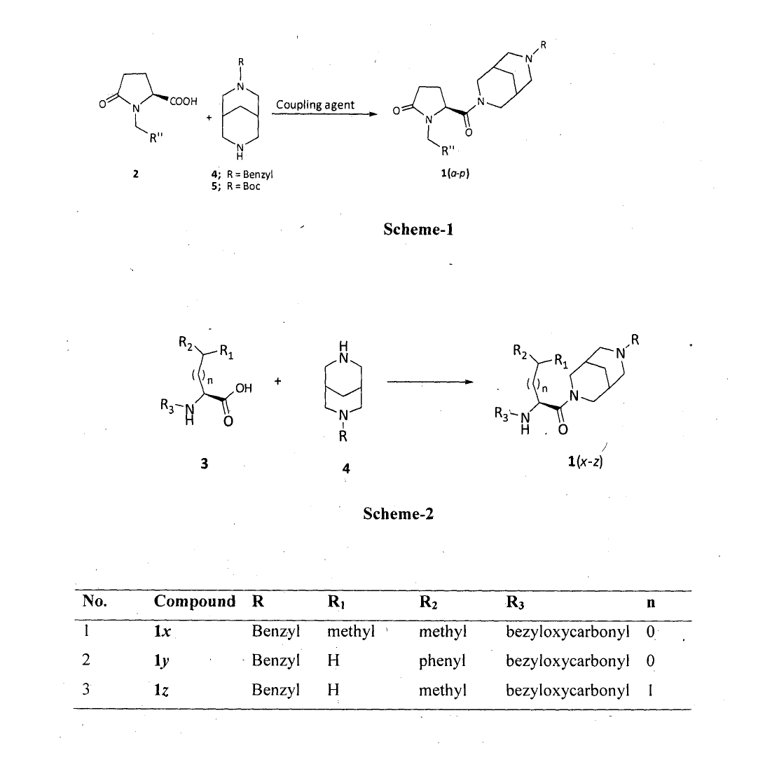

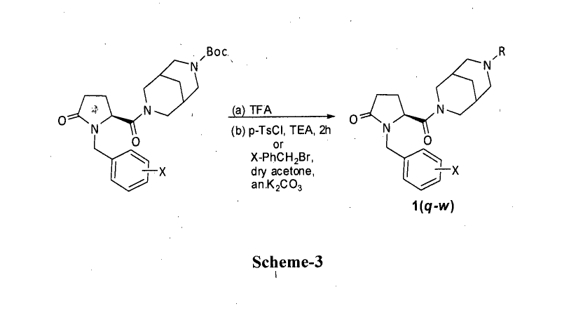

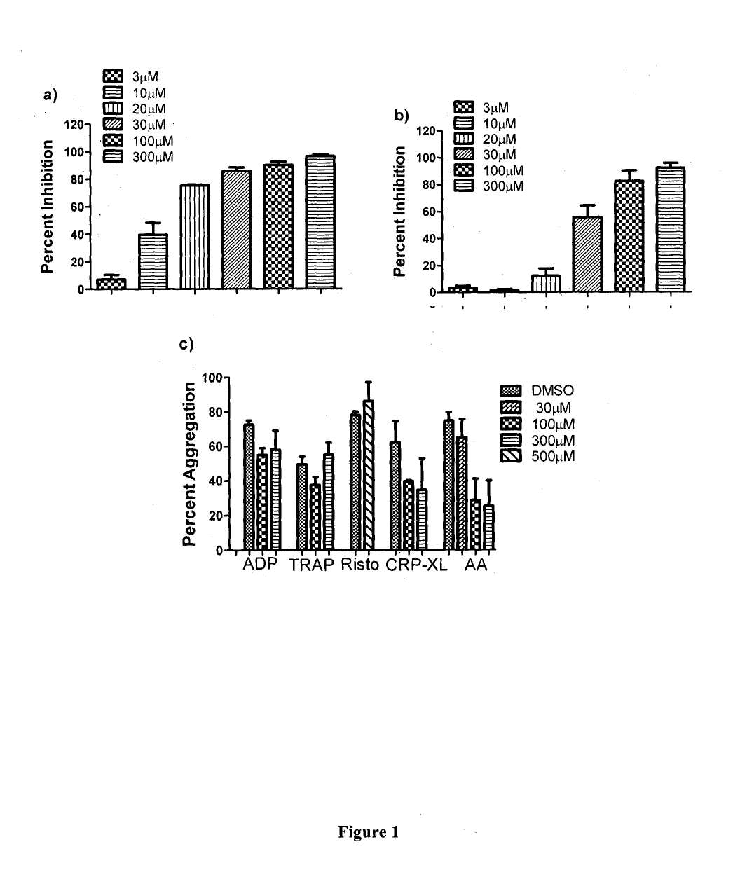

All the molecules were further tested (30 μM, in vitro) for their inhibitory effect on human platelet aggregation induced by various agonists (in vitro). The compounds 1d, 1g, 1h, 1o, 1u, 1v and 1w exhibited significant inhibition against collagen induced platelet aggregation (TABLE 1). Compound 1d, 1g, 1h, 1u, 1v and 1w is exhibiting dose dependent anti-platelet efficacy through dual mechanism inhibited both collagen as well as U46619 (thromboxane receptor agonist) induced platelet aggregation. Compound 1d was the most potent among these groups and exhibited a percent inhibition of 86±3.41% against collagen. The compound 1d, even up to 300 μM, did not exhibit any significant effect against ADP, thrombin mimetic SFLLRN (TRAP), GPVI agonist collagen related peptide (CRP-XL) and GP 1b-IX-V agonist Ristocetin induced platelet aggregation. However, the compound at 30 μM displayed a significant inhibition of platelet aggregation induced by thromboxane A₂ analog U46619 (75.5±6%). The compound 1d did not exhibit any inhibition of COX pathway via arachidonic acid induced platelet aggregation at 30 μM, but at higher concentration (300 μM and 500 μM) the compound 1d attenuated platelet aggregation upto 50%. These findings indicate that the compound 1d might exhibit its anti-platelet efficacy through dual mechanism, and hence requires further confirmation regarding its mechanism of action. Since aspirin is already proven clinically for its inhibitory effect on the production of thromboxane A2 by inhibiting cyclooxygenase, hence these compounds having a relatively potent TP-receptor as well as collagen receptor antagonistic activity could be very useful as therapeutic antithrombotic agents. Moreover, the action of compound 1d is platelet specific, since its presence did not altered the coagulability of blood as assessed by TT, PT and aPTT in human plasma. (FIG. 1)

TABLE 1
In vivo (% protection; inducer, collagen + epinephrine) and in vitro
(% inhibition of aggregation; inducer, collagen) activity of bispidine
derivatives of N-substituted pyroglutamic acid, 1(a-w).

| No. | Compound | R | R” | Protection (%) * | Inhibition (%) ^{#,δ} |
|---|---|---|---|---|---|
| 1 | 1a | Boc | Phenyl | 40 | 06.00 ± 14.00 |
| 2 | 1b | Boc | 2-Bromophenyl | 50 | Ns |
| 3 | 1c | Benzyl | Phenyl | 30 | 44.00 ± 13.00 |
| 4 | 1d | Benzyl | 2-Bromophenyl | 40 | 86.00 ± 3.41 |
| 5 | 1e | Boc | 4-Methylphenyl | 40 | 25.00 ± 9.00 |
| 6 | 1f | Benzyl | 4-Methylphenyl | 25 | 07.00 ± 7.00 |
| 7 | 1g | Benzyl | 2,6-Dichlorophenyl | 40 | 68.00 ± 6.00 |
| 8 | 1h | Benzyl | 4-Chlorophenyl | 30 | 52.00 ± 8.00 |
| 9 | 1i | Benzyl | Tosyl | 30 | 29.00 ± 1.00 |
| 10 | 1j | Boc | 4-Cyanophenyl | 55 | 11.00 ± 3.00 |
| 11 | 1k | Boc | 4-Chlorophenyl | 30 | 06.00 ± 11.00 |
| 12 | 1l | Boc | 2,6-dichlorophenyl | 30 | 03.00 ± 3.00 |
| 13 | 1m | Boc | 4-Methoxyphenyl | 45 | 10.00 ± 3.00 |
| 14 | 1n | Boc | 1-Naphthyl | 30 | 11.00 ± 4.00 |
| 15 | 1o | Benzyl | 4-Bromophenyl | 40 | 57.00 ± 11.00 |
| 16 | 1p | Benzyl | 4-Methoxyphenyl | 30 | 10.00 ± 4.00 |
| 17 | 1q | Benzoyl | 2-Bromophenyl | 60 | 25.60 ± 3.55 |
| 18 | 1r | Tosyl | 2-Bromophenyl | 55 | 16.80 ± 5.66 |
| 19 | 1s | Benzoyl | 4-Methylphenyl | 40 | 20.50 ± 7.50 |
| 20 | 1t | Tosyl | 4-Methylphenyl | 40 | 37.50 ± 10.50 |
| 21 | 1u | 2-Bromobenzyl | 4-Methylphenyl | 30 | 67.00 ± 10.00 |
| 22 | 1v | 4-Bromobenzyl | 4-Methylphenyl | 50 | 61.00 ± 8.00 |
| 23 | 1w | 2-Chlorobenzyl | 4-Methylphenyl | 35 | 67.00 ± 8.00 |
| | Aspirin | | | 40 (at 170 μm) | — |
| | DMSO | | | | 25.31 ± 2.59 |

* Collagen-epinephrine induced pulmonary thromboembolism in mice (in vivo)
^{#} Inhibition of collagen induced platelet aggregation in human platelets (in vitro)
^{δ} Compound concentration used = 30 μM; n = 3; ns, not significant

Carboxamides of substituted or protected amino acids with substituted bispidines were also prepared 1(x-z) and they exhibited low profile antiplatelet efficacy both in vitro and in vivo (Table-2).

TABLE 2
In vivo (% protection; inducer, collagen + epinephrine) in vitro (% inhibition of
aggregation; inducer, collagen) activity of bispidine derivatives of N-protected amino acids, 1(x-z)

| | | | | | | | Protection | Inhibition |
|---|---|---|---|---|---|---|---|---|
| No. | Compound | R | R₁ | R₂ | R₃ | n | (%)* | (%)^{# δ} |
| 1 | 1x | Benzyl | methyl | methyl | Bezyloxycarbonyl | 0 | 20 | 18.00 ± 04 |
| 2 | 1y | Benzyl | H | phenyl | Bezyloxycarbonyl | 0 | 55 | 22.00 ± 5.00 |
| 3 | 1z | Benzyl | H | methyl | Bezyloxycarbonyl | 1 | 40 | 12.00 ± 10.00 |
| | Aspirin | | | | | | 40 (at | |
| | | | | | | | 170 μm) | |

*Collagen-epinephrine induced pulmonary thromboembolism in mice (in vivo)
^{#}Inhibition of collagen induced platelet aggregation in human platelets (in vitro)
^{δ}Compound concentration used = 30 μM; n = 3; ns, not significant

### Advantages of the Invention

1. Both starting materials L-glutamic acid and 4-piperidone hydrochloride and reaction reagents are economically cheap, easily accessible and non-hazardous in nature.
2. All the products were isolated in moderately good yield (ranging 50 to 90%).
3. All the final products are very much stable even at room temperature.
4. The compounds exhibited tremendous inhibition of % platelet aggregation induced by collagen induced aggregation in human platelets (in vitro) varies from 03.00±3.00 to 86.00±3.41% at 30 μM concentration out of them seven compounds exhibited highly promising anti-platelet efficacy inhibited collagen, in vitro varies from 57.00±11.00 to 86.00±3.41%
5. Moreover, five compound exhibited dose dependent anti-platelet efficacy through dual mechanism inhibited both collagen as well as U46619 (thromboxane receptor agonist) induced platelet aggregation and varies from 52±03 to 85±03.

1. A compound of general formula 1: 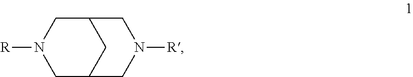
wherein R′ is 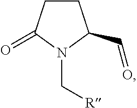
wherein R is selected from the group consisting of a tosyl group, a tert-butyloxycarbonyl group, a benzyl group, a halobenzyl group, and a benzoyl group; and
wherein R″ is selected from the group consisting of a cyano group, a benzyl group, and a napthyl group, and wherein R″ is optionally substituted with a member selected from the group consisting of a halogen, a methyl group, a methoxy group, a cyano group, and a tosyl group;
or
wherein R is benzyl;
wherein R′ is 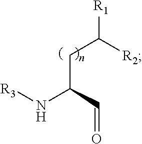
wherein R₁ is hydrogen;
wherein R₂ is selected from the group consisting of a methyl group and an aryl group;
wherein R₃ is a benzyloxycarbonyl group, and
wherein n=0,1.

2. The compound as claimed in claim 1, wherein the compound of general formula 1 is selected from the group consisting of:
tert-butyl 7-(1-Benzyl-5-oxo-pyrrolidine-2-carbonyl)-3,7-diaza-bicyclo nonane-3-carboxylate, (1a);
tert-butyl 7-[1-(2-Bromo-benzyl)-5-oxo-pyrrolidine-2-carbonyl]-3,7-diaza-bicyclo[3.3.1]nonane-3-carboxylate, (1b);
1-Benzyl-5-(7-benzyl-3,7-diaza-bicyclo[3.3.1]nonane-3-carbonyl)-pyrrolidin-2-one, (1c);
(5S)-5-(7-Benzyl-3,7-diaza-bicyclo[3.3.1]nonane-3-carbonyl)-1-(2-bromo-benzyl)-pyrrolidin-2-one, (1d);
tert-butyl 7-[1-(4-Methyl-benzyl)-5-oxo-pyrrolidine-2-carbonyl]-3,7-diaza-bicyclo[3.3.1]nonane-3-carboxylate, (1e);
(5S)-5-(7-Benzyl-3,7-diaza-bicyclo[3.3.1]nonane-3-carbonyl)-1-(4-methyl-benzyl)-pyrrolidin-2-one, (1f);
(5S)-5-(7-benzyl-3,7-diazabicyclo[3.3.1]nonane-3-carbonyl)-1-(2,6-dichlorobenzyl) pyrrolidin-2-one, (1g);
(5S)-5-(7-benzyl-3,7-diazabicyclo[3.3.1]nonane-3-carbonyl)-1-(4-chlorobenzyl)pyrrolidin-2-one, (1h);
tert-butyl 7-((S)-1-(4-cyanobenzyl)-5-oxopyrrolidine-2-carbonyl)-3,7-diazabicyclo[3.3.1]nonane-3-carboxylate, (1j);
tert-butyl 7-((S)-1-(4-chlorobenzyl)-5-oxopyrrolidine-2-carbonyl)-3,7-diazabicyclo[3.3.1]nonane-3-carboxylate, (1k);
tert-butyl 7-((S)-1-(2,6-dichlorobenzyl)-5-oxopyrrolidine-2-carbonyl)-3,7-diazabicyclo[3.3.1]nonane-3-carboxylate, (1l);
tert-butyl 7-((S)-1-(4-methoxybenzyl)-5-oxopyrrolidine-2-carbonyl)-3,7-diazabicyclo[3.3.1]nonane-3-carboxylate, (1m);
tert-butyl 7-((S)-1-(naphthalen-1-ylmethyl)-5-oxopyrrolidine-2-carbonyl)-3,7-diazabicyclo[3.3.1]nonane-3-carboxylate, (1n);
(5S)-5-(7-benzyl-3,7-diazabicyclo[3.3.1]nonane-3-carbonyl)-1-(4-bromobenzyl)pyrrolidin-2-one, (1o);
(5S)-5-(7-benzyl-3,7-diazabicyclo[3.3.1]nonane-3-carbonyl)-1-(4-methoxybenzyl)pyrrolidin-2-one, (1p);
(5S)-5-(7-Benzoyl-3,7-diaza-bicyclo[3.3.1]nonane-3-carbonyl)-1-(2-bromo-benzyl)-pyrrolidin-2-one, (1q);
1-(2-Bromo-benzyl)-5-[7-(toluene-4-sulphonyl)-3,7-diaza-bicyclo[3.3.1]nonane-3-carbonyl]-pyrrolidin-2-one, (1r);
1-(4-Methyl-benzyl)-5-[7-(toluene-4-sulphonyl)-3,7-diaza-bicyclo[3.3.1]nonane-3-carbonyl]-pyrrolidin-2-one, (1t);
(5S)-5-(7-Benzoyl-3,7-diaza-bicyclo[3.3.1]nonane-3-carbonyl)-1-(4-methyl-benzyl)-pyrrolidin-2-one, (1s);
(5S)-5-(7-(2-bromobenzyl)-3,7-diazabicyclo[3.3.1]nonane-3-carbonyl)-1-(4-methylbenzyl) pyrrolidin-2-one, (1u);
(5S)-5-(7-(4-bromobenzyl)-3,7-diazabicyclo[3.3.1]nonane-3-carbonyl)-1-(4-methylbenzyl) pyrrolidin-2-one, (1v);
(5S)-5-(7-(4-chlorobenzyl)-3,7-diazabicyclo[3.3.1]nonane-3-carbonyl)-1-(4-methylbenzyl) pyrrolidin-2-one, (1w);
benzyl (2S)-1-(7-benzyl-3,7-diazabicyclo[3.3.1]nonan-3-yl)-3-methyl-1-oxobutan-2-yl carbamate, (1x);
benzyl (2S)-1-(7-benzyl-3,7-diazabicyclo[3.3.1]nonan-3-yl)-4-methyl-1-oxopentan-2-yl carbamate, (1z); and
benzyl (2S)-1-(7-benzyl-3,7-diazabicyclo[3.3.1]nonan-3-yl)-1-oxo-3-phenylpropan-2-yl carbamate, (1y).

3. (canceled)

4. (canceled)

5. A process for preparing the compound of general formula 1, as claimed in claim 1, comprising:
reacting a first compound with a second compound to obtain a reaction mass comprising the compound of general formula 1, wherein the first compound is selected from the group consisting of:
(a) a compound of general formula 4: 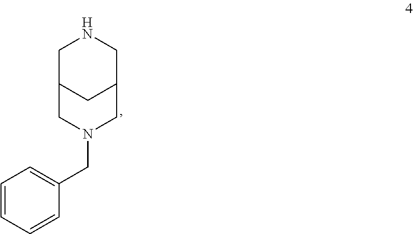
and
(b) a compound of general formula 5: 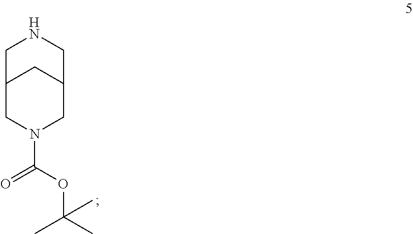
and
wherein the second compound is selected from the group consisting of
(a) a compound of general formula 2: 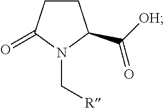
and
(b) a compound of general formula 3: 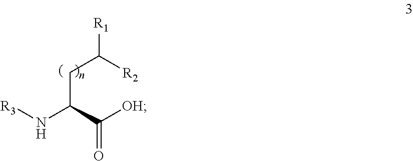
wherein, R″ is selected from the group consisting of a halogen, a cyano group, a lower alkyl group, an aryl group, a substituted aryl group, and a substituted tosyl group;
wherein R1 is selected from the group consisting of hydrogen and a lower alkyl group;
wherein R2 is selected from the group consisting of a lower alkyl and an aryl group;
wherein R3 is selected from the group consisting of tert-butyloxycarbonyl and benzyloxycarbonyl; and
wherein n=0,1;
with the proviso that the compound of general formula 2 is reacted with compounds of general formula 4 or 5 and the compound of general formula 3 is reacted with the compound of general formula 4 only.

6. The process of claim 5, wherein the reacting step takes place in the presence of a coupling agent selected from the group consisting of dicyclohexylcarbodiimide, benzotriazole-1-yl-oxy-tris-pyrrolidino-phosphonium hexafluorophophate, isobutyl chloroformate-TEA/DIPEA, oxalyl chloride-TEA/DIPEA and an activating agent 1-hydroxy benzotrizole at a temperature in the range of −20° C. to 0° C. for a period in the range of 30 to 45 min, followed by stirring at a temperature in the range of 25 to 30° C. for a period in the range of 2 to 3 hours in an aprotic solvent selected from the group consisting of DCM, THF and dioxane.

7.-13. (canceled)

14. The process according to claim 5, wherein the compound of general formula 1 prepared is one or more of: 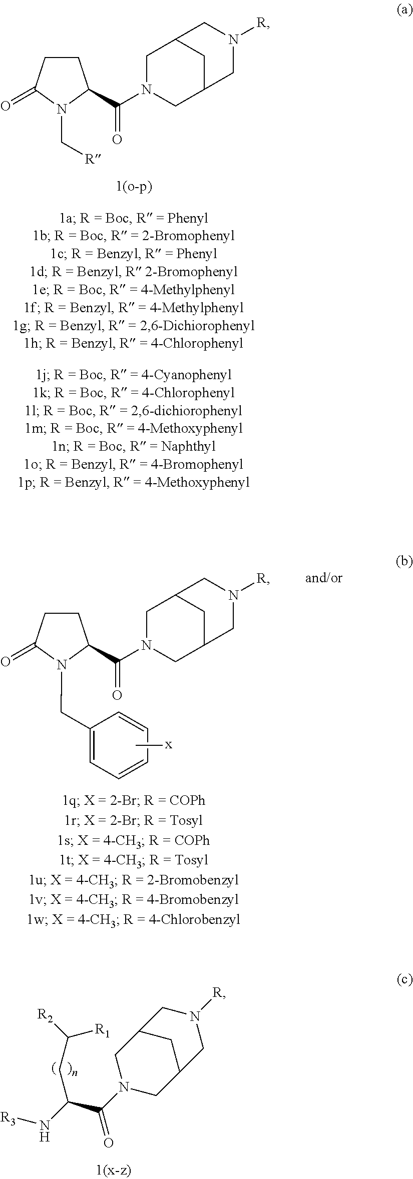
wherein:
R is benzyl;
R1 is selected from the group consisting of hydrogen and a methyl group;
R2 is selected a methyl group;
R3 is a bezyloxycarbonyl group; and
n=0,1;
with the proviso that if the reaction mass thus obtained comprises one or more compounds of formulas 1a, 1b, 1e, and 1j to 1n, then the method further comprising the steps of:
deprotecting a Boc-Group in the reaction mass with TFA at a temperature ranging between 0° C. to 15° C. for a period in the range of 4 to 5 hours, followed by
N-acylation at a temperature in the range of 0° C. to 25° C. in a solvent selected from the group consisting of DCM and THF, followed by
N-benzylation at a temperature in the range of 50 to 60° C. for a period in the range of 4 to 5 hours in acetone to obtain a reaction mass comprising a deprotected compound of formula 1q to 1w, and
converting the deprotected compound of formula 1q to 1w thus obtained by N-benzylation, benzoylation and/or tosylation to provide a protected compound 1q to 1w.

15. The process of claim 14, wherein the N-benzylation is carried out in dry acetone in the presence of anhydrous potassium carbonate (K₂CO₃) followed by the addition of substituted benzyl bromide by refluxing at a temperature in the range of 50-60° C. for 2 to 3 hours.

16. The process of claim 14, wherein the benzoylation is carried out in dry dichloromethane using benzoyl chloride in the presence of triethylamine or diisopropylethyl amine at a temperature in the range of 0 to 5° C. for 30 to 60 minutes.

17. The process of claim 14, wherein the tosylation is carried out in dry dichloromethane using toluenesulphonyl chloride in the presence of triethylamine or diisopropylethyl amine at a temperature in the range of 0 to 5° C. for 30 to 60 minutes.

18. The compound of claim 2, wherein the compounds 1(c-d), 1(f-h), 1(o-p), and 1(u-z) are salts selected from the group consisting of a hydrochloride salt and a tartrate salt.
